(19) United States
(12) Patent Application Publication
(10) Pub. No.: US 2004/0235831 A1
(43) Pub. Date: Nov. 25, 2004

(54) Composition for caring for the hair or the eyelashes, containing a pyrazolecarboxamide compound, use thereof for stimulating the growth of the hair and the eyelashes and/or for reducing their loss

(75) Inventors: Roger Rozot, Lagny S/Marne (FR); Christophe Boulle, Lagny S/Marne (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL, S.A., Paris (FR)

(21) Appl. No.: 10/716,410

(22) Filed: Nov. 20, 2003

Related U.S. Application Data
(60) Provisional application No. 60/428,721, filed on Nov. 25, 2002.

(30) Foreign Application Priority Data
Nov. 20, 2002 (FR) 02/14534

(51) Int. Cl. A61K 31/541; A61K 31/5377; A61K 31/496; A61K 31/454; A61K 31/415

(52) U.S. Cl. 514/227.5; 514/235.5; 514/254.05; 514/326; 514/406

(57) ABSTRACT

The invention relates to a composition for caring for or making up the hair or the eyelashes, which is intended to limit their loss and/or to increase their density, and containing a pyrazole compound of formula (I), or a salt thereof:

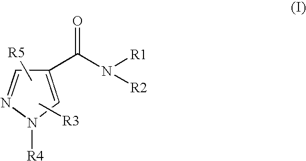

in which:

R₁ and R₂ represent H; an optionally substituted C₁-C₂₀ alkyl; or at least one ring optionally containing at least one hetero atom, these rings possibly being fused, comprising a carbonyl or thiocarbonyl function, and possibly being substituted;

R₃ and R₅ represent H, a halogen, OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, A or at least one ring optionally containing at least one hetero atom, these rings possibly being fused, comprising a carbonyl or thiocarbonyl function and possibly being substituted;

R₄ represents H, A, COR₆, CSR₆, COOR₆, CONR₆R′₆, CSNR₆R′₆, SO₂R₆, SO₂NR₆R′₆ or at least one ring optionally containing a hetero atom, these rings possibly being fused, comprising a carbonyl or thiocarbonyl function, and possibly being substituted;

R₆, R′₆, R″₆ and R′″₆ represent H; an optionally substituted C₁-C₂₀ alkyl; at least one ring optionally containing at least one hetero atom, these rings possibly being fused, comprising a carbonyl or thiocarbonyl function, and possibly being substituted with at least one substituent R;

A represents an optionally substituted C₁-C₂₀ alkyl.

### FIELD OF THE INVENTION

The invention relates to a care or makeup composition for keratin fibres, especially human keratin fibres, containing an effective amount of a pyrazole compound and more especially of a pyrazolecarboxamide compound, which is intended to induce and/or stimulate the growth of keratin fibres and/or to reduce their loss. The invention also relates to a cosmetic treatment process for stimulating the growth of keratin fibres and/or reducing their loss.

The human keratin fibres to which the invention applies are especially head hair, the eyebrows, the eyelashes, beard hair, moustache hair and pubic hairs. The invention applies more especially to human head hair and/or eyelashes.

In particular, the invention relates to a composition for caring for or making up the hair or the eyelashes, containing an effective amount of a pyrazolecarboxamide compound, which is intended to increase their density and/or to improve their appearance.

### BACKGROUND OF THE INVENTION

Hair growth and hair renewal are mainly determined by the activity of the hair follicles and of their matrix environment. Their activity is cyclical and comprises essentially three phases, namely the anagenic phase, the catagenic phase and the telogenic phase.

The anagenic phase (active phase or growth phase), which lasts several years and during which the hair gets longer, is followed by a very short and transient catagenic phase which lasts a few weeks. During this phase, the hair undergoes a change, the follicle becomes atrophied and its dermal implantation appears higher and higher.

The terminal phase or telogenic phase, which lasts a few months, corresponds to a resting phase of the follicle and the hair ends up by falling out. At the end of this rest period, a new follicle is regenerated in situ and another cycle begins.

The head of hair is thus under permanent renewal, and, out of the approximately 150,000 hairs that make up a head of hair, about 10% are at rest and will be replaced within a few months.

The natural loss or falling-out of the hair may be estimated, on average, as being a few hundred hairs per day for a normal physiological state. This process of permanent physical renewal undergoes a natural change during ageing: the hairs become finer and their cycles shorter.

In addition, various causes may result in a substantial, temporary or permanent loss of hair. This may be loss and impairment of hair at the terminal stage of pregnancy (post-partum), during states of dietary malnutrition or imbalance, or during states of asthenia or of hormonal dysfunction, as may be the case during or at the terminal stage of the menopause. It may also be a case of loss or impairment of the hair related to seasonal phenomena.

It may also be a matter of alopecia, which is essentially due to a disturbance in hair renewal, resulting, in a first stage, in acceleration of the frequency of the cycles to the detriment of the quality of the hair, and then of their quantity. The successive growth cycles result in hairs that are finer and finer and shorter and shorter, gradually transforming into an unpigmented down, thus resulting in a progressive impoverishment of the head of hair. Certain areas are preferentially affected, especially the temporal or frontal lobes in men, and a diffuse alopecia of the crown of the head in women.

The term alopecia also covers a whole family of afflictions of hair follicles whose final consequence is the permanent, partial or general loss of the hair. This is more particularly a matter of androgenic alopecia. In a large number of cases, early loss of hair occurs in genetically predisposed individuals; this is then a matter of androchronogenetic alopecia. This form of alopecia especially affects men.

It is moreover known that certain factors, such as hormonal imbalance, physiological stress or malnutrition, can accentuate the phenomenon.

In certain dermatoses of the scalp with an inflammatory component, for instance psoriasis or seborrhoeic dermatitis, hair loss may be greatly accentuated or may result in highly disrupted follicular cycles.

The cosmetics and pharmaceutical industries have for many years been investigating compositions for eliminating or reducing alopecia, and especially for inducing or stimulating hair growth or reducing its loss.

In this perspective, a large number of compositions comprising very diverse active agents have already been proposed, for instance 2,4-diamino-6-piperidinopyrimidine 3-oxide, or “minoxidil”, described in patents U.S. Pat. No. 4,139,619 and U.S. Pat. No. 4,596,812, or the numerous derivatives thereof such as those described, for example, in documents EP 0 353 123, EP 0 356 271, EP 0 408 442, EP 0 522 964, EP 0 420 707, EP 0 459 890 and EP 0 519 819.

Clinical studies have shown that PGF2-α analogues have the property of inducing the growth of body hairs and eyelashes in man and animals (Murray A. and Johnstone M. D., 1997, Am. J. Opht., 124(4), 544-547). In man, tests performed on the scalp have shown that a prostaglandin E2 analogue (viprostol) has the property of increasing the hair density (Roenigk H. H., 1988, Clinic Dermatol., 6(4), 119-121).

Moreover, document WO 98/33497 describes pharmaceutical compositions containing prostaglandins or prostaglandin derivatives, for combating hair loss in man. Prostaglandins of the type A2, F2α and E2 are mentioned as being preferred.

However, prostaglandins are molecules with a very short biological half-life, which act in an autocrine or paracrine manner, this reflecting the local and labile nature of the metabolism of prostaglandins (Narumiya S. et al., 1999, Physiol. Rev., 79(4), 1193-1226).

It is thus seen to be important, in order to maintain and/or increase the hair density in man, to preserve the endogenous reserves of PGF2-α and similarly of PGE2 in various compartments of the hair follicle or its immediate cutaneous environment.

One solution that gives good results is the use of lipoxygenase-inhibiting compounds and/or cyclooxygenase-inducing compounds to promote hair growth; one theory is that the use of such compounds directs the metabolism of fatty acids towards the endogenous synthesis of prostaglandins in preference to other routes.

However, to further improve the results, it would be desirable to be able to prolong the activity of the prostaglandins involved in growing the hair and keeping it alive.

It is moreover well known that the programmes of differentiation of the keratinocytes of the epidermis and of the hair follicle are clearly different. Thus, it is known that the keratins of the hair stalk represent a family (Langbein et al., 2001, J. Biol. Chem. 276: 35123-35132) that is different from the one expressed in the epidermis, that differentiation markers such as the keratins K₁ and K₁₀ are not expressed in the hair follicle and in particular in the outer sheath (Lenoir et al., 1988, Dev. Biol. 130: 610-620), that trichohyalin (O'Guin et al., 1992, J. Invest. Dermatol. 98: 24-32) and keratin K6irs (Porter et al., 2001, Br. J. Dermatol. 145: 558-568) are expressed in the hair follicle, in particular in the inner sheath, but not in the epidermis, and that type-1 cyclooxygenase, although expressed in the epidermis, is not expressed in the keratinocytes of the hair follicle but in the dermal papilla (Michelet. et al., 1997, J. Invest. Dermatol. 108: 205-209).

Surprisingly, the Applicant has now demonstrated that an enzyme specifically involved in the degradation of these prostaglandins is present in the dermal papilla of the hair, which is a compartment that is a decisive factor in the life of the hair. Specifically, the Applicant has now proven the presence of 15-hydroxyprostaglandin dehydrogenase (abbreviated as 15-PGDH) at this level. The Applicant has also shown that the inhibition of 15-PGDH has a beneficial effect on hair growth.

Consequently, the present invention relates to a care or treatment composition for keratin fibres, and especially hair fibres, containing at least one particular inhibitor of 15-hydroxyprostaglandin dehydrogenase and a physiologically acceptable medium.

15-PGDH is a key enzyme in the deactivation of prostaglandins, in particular of PGF2-α and PGE2, which are important mediators of hair growth and survival. It corresponds to the classification EC 1.1.1.141 and is NAD⁺-dependent. It has been isolated from pig kidney; its inhibition with a thyroid hormone, triiodothyronine, at doses very much higher than the physiological doses, has especially been observed.

However, it has never been proposed to use a 15-PGDH inhibitor to maintain and/or increase the density of human keratin fibres and especially the hair density and/or to reduce the heterogeneity of the diameters of the keratin fibres and especially of the hair in man. The expression “increase the density of human keratin fibres, and especially the hair density” means increasing the number of keratin fibres, and especially of hairs per cm² of skin or of scalp.

Pyrazolecarboxamide compounds necessarily comprising an optionally substituted phenyl radical on the nitrogen of the amide group and having anti-inflammatory properties but absolutely no 15-PGDH-inhibitory properties, are moreover known from document EP 1 176 140.

In addition, patent U.S. Pat. No. 4,251,658 describes pyrazole compounds whose chemical structure is different from that of the pyrazole compounds to which the invention applies. In particular, the amide group is not present in the compounds described in the said patent. Furthermore, these compounds are described as 15-PGDH-inhibitors obtained from pig lung rather than from the skin (external organ), especially human skin.

However, there is nothing to say that the 15-PGDH present in pig lung is the same as that present in human skin, and that a compound presented as a 15-PGDH inhibitor from pig lung will also inhibit the 15-PGDH present in the human dermis and especially in the dermal papilla of the hair.

Finally, pyrazole compounds in which the carboxamide group is branched in position 4 instead of being branched in position 5 of the pyrazole ring are known from document WO 03/043983. In addition, V always represents a substituted phenyl, which is not envisaged in the compounds to which the present invention applies.

### SUMMARY OF THE INVENTION

The Applicant has found that certain pyrazole compounds, and especially certain salified or non-salified pyrazolecarboxamides, are, surprisingly, endowed with favourable activity towards improving the density of human keratin fibres and especially of the hair. The Applicant has moreover found that these compounds are inhibitors of 15-hydroxyprostaglandin dehydrogenase.

One subject of the present invention is thus a composition for caring for and/or making up keratin fibres, especially human keratin fibres, containing, in a physiologically acceptable medium, an effective amount of a pyrazole compound of formula (I), or a salt thereof:

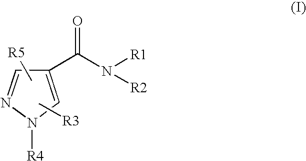

in which:

R₁ and R₂ are chosen independently from:

hydrogen,

saturated or unsaturated, linear or branched C₁-C₂₀ alkyl radicals optionally substituted with at least one substituent T₁,

saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;

R₃ and R₅ are chosen independently from:

hydrogen,

A,

halogens,

the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆,

saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;

R₄ is chosen from:

hydrogen,

A,

the groups COR₆, CSR₆, COOR₆, CONR₆R′₆, CSNR₆R′₆, SO₂R₆, SO₂NR₆R′₆,

saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;

R₆, R′₆, R″₆ and R′″₆ are chosen from:

hydrogen,

saturated or unsaturated, linear or branched C₁-C₂₀ alkyl radicals optionally substituted with at least one substituent R′,

saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;

R is chosen from:

saturated or unsaturated, linear or branched C₁-C₂₀ alkyl radicals,

halogens,

the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇;

R′ is chosen from:

saturated or unsaturated, linear or branched C₁-C₂₀ alkyl radicals,

halogens,

the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,

saturated or unsaturated rings, of 4 to 7 atoms, optionally containing at least one hetero atom chosen from O, N and S, these rings possibly being fused and/or comprising a carbonyl or thiocarbonyl function;

R₇, R′₇, R″₇ and R′″₇ independently represent hydrogen or a saturated or unsaturated, linear or branched C₁-C₂₀ alkyl;

A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;

T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R.

The invention also relates to the use of at least one pyrazole compound of formula (I) or a salt thereof, as defined above, as an agent for inducing and/or stimulating the growth of keratin fibres, especially human keratin fibres such as human eyelashes and hair, and/or for reducing their loss and/or for increasing their density.

The invention also applies to the keratin fibres of mammalian animals (for example dogs, horses or cats).

The invention also relates to the cosmetic use of at least one pyrazole compound of formula (I), or a salt thereof, in a cosmetic care and/or makeup composition for human keratin fibres to induce and/or stimulate their growth, to reduce their loss and/or to increase their density, and also to the use of at least one compound of formula (I), or a salt thereof, for the preparation of a care or treatment composition for human keratin fibres, which is intended to induce and/or stimulate the growth of the fibres and/or to reduce their loss and/or to increase their density.

The human keratin fibres to which the invention applies are especially head hair, the eyebrows, the eyelashes, beard hair, moustache hair and pubic hair. The invention applies more especially to human head hair and/or eyelashes.

The invention also relates to the cosmetic use of at least one pyrazole compound of formula (I), or a salt thereof, in a human cosmetic haircare composition for reducing hair loss and/or for increasing its density. A subject of the invention is also the use of at least one pyrazole compound of formula (I), or a salt thereof, for the preparation of a human hair composition, which is intended to induce and/or stimulate hair growth and/or reduce its loss and/or increase its density.

In particular, the invention relates to the cosmetic use of at least one pyrazole compound of formula (I), or a salt thereof, in a human cosmetic haircare composition or for the preparation of a human hair composition for treating or which is intended to treat alopecia of natural origin and in particular androgenic or androchronogenetic alopecia. Thus, this composition makes it possible to keep the head of hair in good condition and/or to combat natural hair loss of humans.

A subject of the invention is also the cosmetic use of at least one pyrazole compound of formula (I), or a salt thereof, in a cosmetic care and/or makeup composition for human eyelashes, to induce and/or stimulate the growth of the eyelashes and/or to increase their density, and also the use of at least one compound of formula (I), or a salt thereof, for the preparation of a care and/or treatment composition for human eyelashes, which is intended to induce and/or stimulate the growth of the eyelashes and/or to increase their density. This composition thus makes it possible to keep the eyelashes in good condition and/or to improve their condition and/or appearance.

A subject of the invention is also a cosmetic process for treating keratin fibres (especially hair or eyelashes) and/or the skin from which the said fibres emerge, including the scalp and the eyelids, which is intended in particular to stimulate the growth of human keratin fibres and/or to reduce their loss, characterized in that it consists in applying to the keratin fibres and/or the skin from which the said fibres emerge a cosmetic composition comprising an effective amount of at least one compound of formula (I), or a salt thereof, leaving this composition in contact with the keratin fibres and/or the skin from which the said fibres emerge, and optionally rinsing the fibres and/or the said skin.

This treatment process has the characteristics of a cosmetic process in that it makes it possible to improve the aesthetics of keratin fibres, and especially of hair and eyelashes, by giving them greater vigour and an improved appearance. In addition, it may be used daily for several months, without medical prescription.

A subject of the invention is also the use of at least one pyrazole compound of formula (I) or of a salt thereof as an inhibitor of the 15-hydroxyprostaglandin dehydrogenase of human skin. A subject of the invention is also the use of at least one pyrazole compound of formula (I) or a salt thereof for the manufacture of a composition for treating disorders associated with 15-hydroxyprostaglandin dehydrogenase in man.

Thus, a subject of the present invention is also a cosmetic process for treating the hair and/or the scalp, which is intended to stimulate the growth of human hair and/or to reduce its loss, characterized in that it consists in applying to the hair and/or the scalp a cosmetic composition comprising an effective amount of at least one compound of formula (I), or a salt thereof, leaving the composition in contact with the hair and/or the scalp, and optionally rinsing the hair and/or the scalp.

More especially, a subject of the present invention is a cosmetic care process for human hair and/or the scalp, to improve their condition and/or appearance, characterized in that it consists in applying to the hair and/or the scalp a cosmetic composition comprising an effective amount of at least one compound of formula (I), or a salt thereof, leaving the composition in contact with the hair and/or the scalp and optionally rinsing the hair and/or the scalp.

A subject of the invention is also a cosmetic care and/or makeup process for human eyelashes, to improve their condition and/or appearance, characterized in that it consists in applying to the eyelashes and/or the eyelids a mascara composition comprising at least one compound of formula (I), or a salt thereof, and leaving the composition in contact with the eyelashes and/or the eyelids. This mascara composition may be applied alone or as a basecoat for a standard pigmented mascara, and may be removed like a standard pigmented mascara.

A subject of the invention is also a care or makeup composition for keratin fibres, comprising, in a physiologically acceptable medium, in particular a cosmetic medium, at least one compound of formula (I), or a salt thereof, and at least one additional active agent for promoting the regrowth of human keratin fibres and/or for limiting their loss, chosen from aminexil, FP receptor agonists and vasodilators, and more especially chosen from aminexil, minoxidil, latanoprost, butaprost and travoprost.

A subject of the invention is also the use of at least one compound of formula (I), or a salt thereof, for the manufacture of a composition for preserving the amount and/or activity of prostaglandins in the hair follicle.

A subject of the invention is also the cosmetic use of at least one compound of formula (I), or a salt thereof, in a cosmetic composition, as an agent for preserving the amount and/or activity of the prostaglandins in the hair follicle.

A subject of the invention is also novel pyrazolecarboxamide compounds of formula III, or a salt thereof:

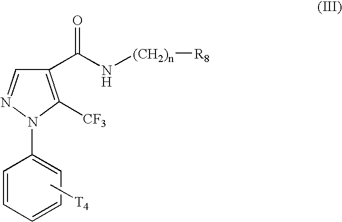

in which R₈ represents OH or —S—(CH₂)ₘ—R₉, with R₉ representing H or Hy; T₄ represents H or 4-COOH; n represents an integer ranging from 1 to 10 and m represents an integer ranging from 1 to 10; Hy represents a heterocycle of 4 to 7 atoms.

### DETAILED DESCRIPTION OF THE EMBODIMENTS OF THE INVENTION

The term “15-hydroxyprostaglandin dehydrogenase inhibitor” means a compound of formula (I) that is capable of inhibiting or reducing the activity of the enzyme 15-PGDH, especially in man, and/or capable of inhibiting, reducing or slowing down the reaction catalysed by this enzyme.

According to one advantageous embodiment of the invention, the compound of formula (I) is a specific inhibitor of 15-PGDH; the term “specific inhibitor” means a compound of formula (I) that has little or no inhibitory effect on the synthesis of prostaglandins, in particular on the synthesis of PGF2-α or PGE2. According to one particular embodiment of the invention, the 15-PGDH inhibitor has little or no inhibitory effect on the synthesis of prostaglandins, in particular on the synthesis of PGF2-α or PGE2. According to one particular embodiment of the invention, the 15-PGDH inhibitor has little or no inhibitory effect on prostaglandin synthase (PGF synthase).

Specifically, the Applicant has now found that PGF synthase is also expressed in the dermal papilla. Maintaining an effective amount of prostaglandins at the site of action thus results from a complex biological equilibrium between the synthesis and degradation of these molecules. The exogenous supply of compounds that inhibit catabolism will therefore be less effective if this activity is combined with an inhibition of the synthesis.

Advantageously, the compounds of formula (I), in salified or non-salified form, show inhibitory activity on 15-PGDH that is higher than the inhibitory activity on PGF synthase. In particular, the ratio between the inhibitory activities on PGF synthase and on 15-PGDH, respectively, for a given concentration, determined especially by the concentrations that inhibit 50% of the enzymatic activity, respectively, of PGF synthase, IC_{50fs}, and of 15-PGDH, IC_{50dh}, is at least greater than 1, especially at least 3:1 and advantageously greater than or equal to 5:1. The preferred compounds of the invention have an IC_{50fs}/IC_{50dh} ratio of greater than or equal to 10:1 and in particular greater than or equal to 15.

In the text hereinbelow, and unless specifically mentioned, the use of the term “compound of formula (I)” should be understood as meaning not only the compound of formula (I) in acidic or basic form but also a salt thereof.

According to the invention, the term “at least one” means one or more (2, 3 or more). In particular, the composition may contain one or more compounds of formula (I). This or these compound(s) may be cis or trans or Z or E isomers, or a mixture of cis/trans or Z/E isomers. This or these compound(s) may also be in tautomeric form. They may also be enantiomers and/or diastereoisomers or a mixture of these isomers, in particular a racemic mixture.

According to the invention, the rings used for R₁, R₂, R₃, R₄, R₅, R₆, R₆′, R₆″, R₆″′, R′, T₁ and T₅ contain from 4 to 7 atoms and better still 5 or 6 atoms. They may be saturated or unsaturated and may optionally comprise one or more hetero atoms such as S, N or O or combinations thereof. As saturated carbon-based rings that may be used, mention may be made of the cyclopentyl or cyclohexyl radical. Heterocycles that may be mentioned include pyridine, piperidine, morpholine, pyrrole, furan, thiophene, imidazole, oxazole, thiazole, pyrazole, pyrimidine, pyrazine and pyridazine rings. An unsaturated carbon-based ring that may be mentioned is the phenyl radical. In addition, these rings may be substituted, in particular with a substituent such as A or R. Furthermore, R₁ and R₂ may form a heterocycle with the nitrogen to which they are attached, containing from 4 to 7 atoms and better still 5 or 6 atoms, and containing from 1 to 3 hetero atoms chosen from O, N and S.

For R₄, heterocycles that may be used include the pyridine, piperidine, morpholine, pyrrole, furan, thiophene, imidazole, oxazole, thiazole, pyrazole, pyrimidine or pyrazine ring.

Furthermore, these rings (or heterocycles) may be alone or fused to another ring, which may or may not be of the same chemical structure, and may thus form fused rings. Fused rings that may be mentioned include naphthyl, benzofuran, benzothiophene and indole radicals.

For the purposes of the invention, the term “alkyl radical” means a hydrocarbon-based radical which may be linear or branched, and saturated or unsaturated. In particular, the alkyl radical contains from 1 to 10 carbon atoms.

As examples of alkyl radicals that may be used in the invention, mention may be made of methyl, ethyl, isopropyl, n-butyl, tert-butyl, n-hexyl, 2-ethylhexyl, ethylene and propylene radicals.

Halogen atoms that may be used include chlorine, fluorine and bromine atoms, and better still fluorine and chlorine atoms.

According to the invention, the compounds of formula (I) are in isolated form, i.e. in non-polymeric form.

According to one particular embodiment of the invention, the pyrazolecarboxamide compound has the formula (II) below, or a salt thereof:

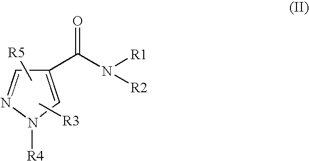

in which:

R₁ and R₂ are chosen independently from:

hydrogen,

saturated or unsaturated, linear or branched C₁-C₂₀ alkyl radicals optionally substituted with at least one substituent T₁, R₁ and R₂ also possibly forming a heterocycle of 4 to 7 atoms with the nitrogen to which they are attached;

R₃ and R₅ are chosen independently from:

hydrogen,

A,

halogens,

the groups OR₆, SR₆, NR₆R′₆, CN, CF₃, COOR₆,

saturated or unsaturated rings of 4 to 7 atoms, optionally containing at least one hetero atom chosen from O, N and S, these rings possibly being fused and/or possibly being substituted with at least one substituent T₃ chosen from A and R;

R₄ is chosen from:

hydrogen,

A,

the groups COR₆ and COOR₆,

saturated or unsaturated hydrocarbon-based rings of 4 to 7 atoms, these rings possibly being substituted with at least one substituent T₄ chosen from A and R;

R₆ and R′₆ are chosen from:

hydrogen,

saturated or unsaturated, linear or branched C₁-C₂₀ alkyl radicals optionally substituted with at least one substituent R′,

saturated or unsaturated rings of 4 to 7 atoms, optionally containing at least one hetero atom chosen from O, N and S, these rings possibly being fused and/or possibly being substituted with at least one substituent R;

R is chosen from:

saturated or unsaturated, linear or branched C₁-C₂₀ alkyl radicals,

halogens,

the groups OR₇, SR₇, NR₇R′₇, CN, CF₃ and COOR₇;

R′ is chosen from:

saturated or unsaturated, linear or branched C₁-C₂₀ alkyl radicals,

halogens,

the groups OR₇, SR₇, NR₇R′₇, CN, CF₃ and COOR₇,

saturated or unsaturated rings of 4 to 7 atoms, optionally containing at least one hetero atom chosen from O, N and S, these rings possibly being fused;

R₇ and R′₇ independently represent hydrogen or a saturated or unsaturated, linear or branched C₁-C₂₀ alkyl radical;

A represents a saturated or unsaturated, linear or branched C₁-C₂₀ alkyl radical optionally substituted with at least one substituent T₅ chosen from halogens, the groups OR₇, SR₇, NR₇R′₇, CN, CF₃ and COOR₇ and saturated or unsaturated rings of 4 to 7 atoms optionally containing at least one hetero atom chosen from O, N and S, these rings possibly being fused and/or possibly being substituted with at least one substituent R;

T₁ is chosen from OR₆, SR₆, NR₆R′₆, CN, CF₃ and COOR₆, halogens, saturated or unsaturated rings of 4 to 7 atoms optionally containing at least one hetero atom chosen from O, N and S, these rings possibly being fused and possibly being substituted with at least one substituent R.

According to one embodiment of the invention, at least one from among R₁ and R₂ represents a saturated C₁-C₂₀ and better still C₁-C₁₀ alkyl radical group substituted with SR₆ or OH. In particular, R₆ represents a C₁-C₂₀ and better still C₁-C₁₀ alkyl radical optionally substituted with a heterocycle Hy of 4 to 7 atoms. For example, at least one from among R₁ and R₂ represents a group (CH₂)ₙR₈ with R₈ representing OH or —S—(CH₂)ₘR₉, with R₉ representing H or Hy, in which n and m each represent an integer ranging from 1 to 20 and better still from 1 to 10. In particular, R₁ represents hydrogen and R₂ represents (CH₂)ₙS(CH₂)ₘR₉, with n being equal to 2 and m being equal to 1. For example, Hy represents a 5-atom heterocycle comprising, for example, oxygen as hetero atom, for instance furan.

Advantageously, at least one from among R₃ and R₅ represents CF₃. In particular, R₃ represents CF₃ and R₅ represents H.

According to one particular embodiment, R₄ represents a hydrocarbon-based ring containing 5 or 6 atoms, in particular an unsaturated ring and especially a phenyl radical optionally substituted with T₄ and, for example, with 4-COOH.

According to one particular embodiment of the invention, the pyrazolecarboxamide compound has the formula (III) below, or a salt thereof:

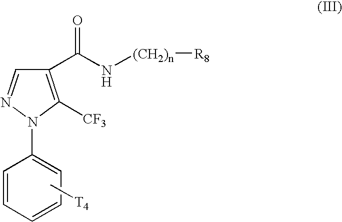

in which R₈ represents OH or —S—(CH₂)ₘ—R₉, with R₉ representing H or Hy; T₄ represents H or 4-COOH; n and m independently represent an integer ranging from 1 to 10 and better still from 1 to 5; Hy representing a heterocycle, especially of 5 or 6 atoms.

According to the invention, the expression “salts of a compound of formula (I)” means the organic or mineral salts of a compound of formula (I).

As mineral salts that may be used according to the invention, mention may be made of the sodium or potassium salts, and also the zinc (Zn²⁺), calcium (Ca²⁺), copper (Cu²⁺), iron (Fe²⁺), strontium (Sr²⁺), magnesium (Mg²⁺), manganese (Mn²⁺) and ammonium salts; hydroxides, carbonates, halides, chlorides, sulphates, phosphates and nitrates.

The organic salts that may be used according to the invention are, for example, the triethanolamine, monoethanolamine, diethanolamine, hexadecylamine, N,N,N′,N′-tetrakis(2-hydroxypropyl)ethylenediamine and tris(hydroxymethyl)aminomethane salts.

The salified or non-salified compounds of formula (I), some of which are known per se, may be manufactured in a known manner and especially as described in the document T. W. Waldrep et al., J. Agr. Food Chem., 1990, 38, 541-544. They are in solid form and especially in pulverulent form.

To the Applicant's knowledge, no prior art document describes or suggests that the pyrazolecarboxamide compounds of formula (I) or the salts thereof have the property of inducing and/or stimulating the growth of human keratin fibres, and in particular the hair and the eyelashes, and/or of reducing their loss, or that these compounds can be used topically to increase the density of the keratin fibres (especially the hair and eyelashes).

The effective amount of a compound of formula (I) or a salt thereof corresponds to the amount required to obtain the desired result (i.e. to increase the density of keratin fibres such as the hair and the eyelashes). A person skilled in the art is thus capable of evaluating this effective amount, which depends on the nature of the compound used, the person on whom it is applied and the time of this application.

In the text hereinbelow, and unless otherwise mentioned, the amounts of the various ingredients in the composition are given as weight percentages relative to the total weight of the composition.

To give an order of magnitude, according to the invention, the compound of formula (I) or a salt thereof, or a mixture of compounds of formula (I) and/or a salt thereof, may be used in an amount representing from 10⁻³% to 10% of the total weight of the composition, preferably in an amount representing from 10⁻³% to 5% and better still from 10⁻²% to 2% of the total weight of the composition, for example from 0.5 to 2%.

The composition of the invention may be for cosmetic or pharmaceutical use. The composition of the invention is preferably for cosmetic use. Thus, the composition must contain a non-toxic, physiologically acceptable medium that can be applied to human skin, including the scalp and the eyelids and to keratin fibres. For the purposes of the invention, the term “cosmetic” means a composition of pleasant appearance, odour and feel.

The compound of formula (I) or a salt thereof may be used in a composition that should be ingested, injected or applied to the skin or to keratin fibres (to any area of skin or fibres to be treated).

According to the invention, the compound of formula (I) may be used orally in an amount of from 0.1 to 300 mg per day, for example from 5 to 10 mg/day.

A preferred composition of the invention is a composition for cosmetic use and in particular for topical application to the skin and keratin fibres, and more especially to the scalp, the hair and the eyelashes.

This composition may be in any known presentation form that is suitable for the mode of use.

For topical application to the skin, the composition may be in the form of an aqueous, alcoholic or aqueous-alcoholic solution or suspension, or an oily suspension, an emulsion of more or less fluid consistency and especially of liquid or semi-liquid consistency, obtained by dispersion of a fatty phase in an aqueous phase (O/W) or conversely (W/O), a solid (O/W) or (W/O) emulsion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or nonionic type. It may thus be in the form of a lotion, a serum, a milk, an O/W or W/O cream, an ointment, a pomade, a balm, a patch or an impregnated pad.

A composition in the form of a foam or alternatively in the form of an aerosol or spray, then comprising a pressurized propellant, may also be envisaged.

In particular, the composition for application to the scalp or the hair may be in the form of a haircare lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for topical application to the eyelashes and body hairs, the composition to which the invention applies may be in the form of a pigmented or unpigmented mascara, to be applied with a brush to the eyelashes or alternatively to beard or moustache hair.

For a composition for use by injection, the composition may be in the form of an aqueous lotion or an oily suspension. For oral use, the composition may be in the form of capsules, granules, drinkable syrups or tablets.

According to one particular embodiment, the composition according to the invention is in the form of a hair cream or hair lotion, a shampoo, a conditioner for the hair or a mascara for the hair or for the eyelashes.

The amounts of the various constituents of the physiological medium of the composition according to the invention are those generally used in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase represents from 5% to 99.9% by weight of the total weight of the composition.

The fatty phase may contain fatty or oily compounds that are liquid at room temperature (25° C.) and atmospheric pressure (760 mm/Hg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

The emulsifiers and co-emulsifiers used to obtain a composition in emulsion form are those generally used in cosmetics and pharmaceuticals. Their nature also depends on the sense of the emulsion. In practice, the emulsifier and, where appropriate, the co-emulsifier are present in the composition in a proportion ranging from 0.1% to 30% by weight, preferably from 0.5 to 20% by weight and better still from 1% to 8% by weight. The emulsion may also contain lipid vesicles and especially liposomes.

When the composition is in the form of an oily solution or gel, the fatty phase may represent more than 90% of the total weight of the composition.

Advantageously, for a hair application, the composition is an aqueous, alcoholic or aqueous-alcoholic solution or suspension and better still a water/ethanol solution or suspension. The alcoholic fraction may represent from 5% to 99.9% and especially from 8% to 80%.

For a mascara application, the composition is a wax-in-water or wax-in-oil dispersion, a gelled oil or an aqueous gel, which may be pigmented or unpigmented.

The composition of the invention may also comprise other ingredients usually used in the fields under consideration, chosen from aqueous-phase or oily-phase solvents, thickeners or gelling agents, dyes that are soluble in the medium of the composition, solid particles such as fillers or pigments, antioxidants, preserving agents, fragrances, electrolytes, neutralizers, film-forming polymers, UV blockers, for instance sunscreens, cosmetic and pharmaceutical active agents with a beneficial effect on the skin and/or keratin fibres, other than the compounds of formula (I) or (II), and mixtures thereof. These additives may be present in the composition in the amounts generally used in cosmetics and dermatology, and especially in a proportion of from 0.01% to 50% and better still from 0.1% to 20%, for example from 0.1% to 10%, relative to the total weight of the composition.

Needless to say, a person skilled in the art will take care to select the optional additional additives and/or the amount thereof such that the advantageous properties of the composition according to the invention, i.e. the inhibition of 15-PGDH in particular or the increase in the density of keratin fibres (hair fibres or eyelashes), are not, or are not substantially, adversely affected by the envisaged addition.

As solvents that may be used in the invention, mention may be made of C₂ to C₈ lower alcohols, for instance ethanol, isopropanol, propylene glycol and certain light cosmetic oils, for instance C₆ to C₁₆ alkanes.

As oils that may be used in the invention, mention may be made of oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, phenyl trimethicone) and fluoro oils (perfluoropolyethers). Waxes that may be mentioned include silicone waxes, beeswax, rice wax, candelilla wax, carnauba wax, paraffin wax and polyethylene wax.

As emulsifiers that may be used in the invention, examples that may be mentioned include glyceryl stearate, glyceryl laurate, sorbitol stearate, sorbitol oleate, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents that may be used in the invention, mention may be made of modified clays, for instance Bentones, metal salts of fatty acids, for instance aluminium stearates, hydrophobic-treated silica and ethylcellulose, and mixtures thereof.

The composition may also contain a cosmetic or pharmaceutical active agent other than the compounds of formula (I), which may be hydrophilic and chosen from proteins, protein hydrolysates, amino acids, polyols, urea, allantoin, sugars and sugar derivatives, water-soluble vitamins, plant extracts (those from Iridacea plants or from soybean) and hydroxy acids (fruit acids or salicylic acid); or lipophilic and chosen from retinol (vitamin A) and its derivatives, especially an ester (retinyl palmitate), tocopherol (vitamin E) and its derivatives (tocopheryl acetate), essential fatty acids, ceramides, essential oils, salicylic acid derivatives, for instance 5-n-octanoyl salicylic acid, hydroxy acid esters, and phospholipids, for instance lecithin, and mixtures thereof.

According to one particular embodiment of the invention, the compound of formula (I) or a salt thereof may be combined with at least one additional active compound that promotes the regrowth and/or limits the loss of keratin fibres (hair or eyelashes). These additional active compounds are chosen especially from the lipoxygenase inhibitors as described in EP 0 648 488, the bradykinin inhibitors described especially in EP 0 845 700, prostaglandins and derivatives thereof, especially those described in WO 98/33497, WO 95/11003, JP 97-100 091 and JP 96-134 242, prostaglandin receptor agonists or antagonists, the non-prostanoic prostaglandin analogues as described in EP 1 175 891, EP 1 175 890, WO 01/74307, WO 01/74313, WO 01/74314, WO 01/74315 or WO 01/72268, and mixtures thereof.

As other additional active compounds that promote the growth of keratin fibres (especially the hair), which may be present in the composition according to the invention, mention may be made of vasodilators, antiandrogens, cyclosporins and analogues thereof, antimicrobial and antifungal agents, anti-inflammatory agents, and retinoids, alone or in a mixture.

The vasodilators that may be used are especially potassium-channel agonists, including minoxidil, and also the compounds described in patents U.S. Pat. Nos. 3,382,247, 5,756,092, 5,772,990, 5,760,043, 5,466 694, 5,438,058 and 4,973,474, cromakalim, nicorandil and diaxozide, alone or in combination.

The antiandrogens that may be used especially include steroidal and non-steroidal 5α-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in patents U.S. Pat. Nos. 5,411,981, 5,565,467 and 4,910,226.

The antimicrobial or antifungal compounds may be chosen from selenium derivatives, octopirox, triclocarban, triclosan, zinc pyrithione, itraconazole, asiatic acid, hinokitiol, mipirocine, tetracyclines, especially erythromycin and the compounds described in EP 0 680 745, clinycin hydrochloride, benzoyl peroxide or benzyl peroxide, minocycline and compounds belonging to the imidazole class, such as econazole, ketoconazole or miconazole or salts thereof, nicotinic acid esters, especially including tocopheryl nicotinate, benzyl nicotinate and C₁-C₆ alkyl nicotinates, for instance methyl or hexyl nicotinate.

The anti-inflammatory agents may be chosen from steroidal anti-inflammatory agents, for instance glucocorticoids, corticosteroids (for example: hydrocortisone) and non-steroidal anti-inflammatory agents, for instance glycyrrhetinic acid and α-bisabolol, benzydamine, salicylic acid and the compounds described in EP 0 770 399, WO 94/06434 and FR 2 268 523.

The retinoids may be chosen from isotretinoin, acitretin and tazarotene.

As other active compounds for promoting the growth and/or limiting the loss of keratin fibres (especially the hair) that may be used in combination with the compound of formula (I), mention may be made of aminexil, 6-0-[(9Z,12Z)octadeca-9,12-dienoyl]hexapyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted aryl ethylenes, N-acylamino acids, flavonoids, ascomycin derivatives and analogues, histamine antagonists, saponins, proteoglycanase inhibitors, oestrogen agonists and antagonists, pseudoterines, cytokines, growth factor promoters, IL-1 or IL-6 inhibitors, IL-10 promoters, TNF inhibitors, benzophenones, hydantoin, retinoic acid; vitamins, for instance vitamin D, vitamin B12 analogues and pantothenol; triterpenes, for instance ursolic acid and the compounds described in U.S. Pat. No. 5,529,769, U.S. Pat. No. 5,468,888 and U.S. Pat. No. 5,631,282; antipruriginous agents, for instance thenaldine, trimeprazine or cyproheptadine; antiparasitic agents, in particular metronidazole, crotamiton or pyrethroids; calcium antagonists, for instance cinnarizine, diltiazem, nimodipine, verapamil, alverine and nifedipine; hormones such as oestriol or its analogues, thyroxine and its salts, and progesterone; FP receptor (type-F prostaglandin receptor) agonists such as latanoprost, bimatoprost, travoprost or unoprostone; mixtures thereof.

Advantageously, the composition according to the invention will comprise at least one 15-PGDH inhibitor as defined above and at least one prostaglandin or prostaglandin derivative, for instance the prostaglandins of series 2 especially including PGF2-α and PGE2 in salt or ester form (for example the isopropyl esters), derivatives thereof, for instance 16,16-dimethyl PGE2, 17-phenyl PGE2, 16,16-dimethyl PGF2-α, 17-phenyl PGF2-α, prostaglandins of series 1, for instance 11-deoxyprostaglandin E1, 1-deoxyprostaglandin E1 in salt or ester form, analogues thereof, especially latanoprost, travoprost, fluprostenol, cloprostenol, viprostol, butaprost, misoprostol, and the salts or esters thereof.

The composition preferably contains at least one non-prostanoic EP2 and/or EP4 receptor agonist as described especially in EP 1 175 892.

It may also be envisaged for the composition comprising at least the compound of formula (I), salified or non-salified, to be in liposomal form, as described especially in document WO 94/22468. Thus, the compound encapsulated in the liposomes may be delivered selectively to the hair follicle.

The composition according to the invention may be applied to the alopecic areas of the scalp and the hair of an individual, and optionally left in contact for several hours and optionally rinsed off.

The composition containing an effective amount of a compound of formula (I), salified or non-salified, may, for example, be applied in the evening, kept in contact throughout the night and optionally shampooed out in the morning. These applications may be repeated daily for one or more months according to the individual.

Advantageously, in the process according to the invention, between 5 and 500 μl of a solution or composition as defined above, comprising from 0.001% to 5% of 15-PGDH inhibitor, are applied to the areas of the scalp to be treated.

### EXAMPLES

Examples of implementation of the invention, which cannot in any way limit its scope, will now be given for illustrative purposes.

### Examples 1 to 8

As examples of pyrazole compounds of formula (I) that may be used in the invention, mention may be made of the following compounds:

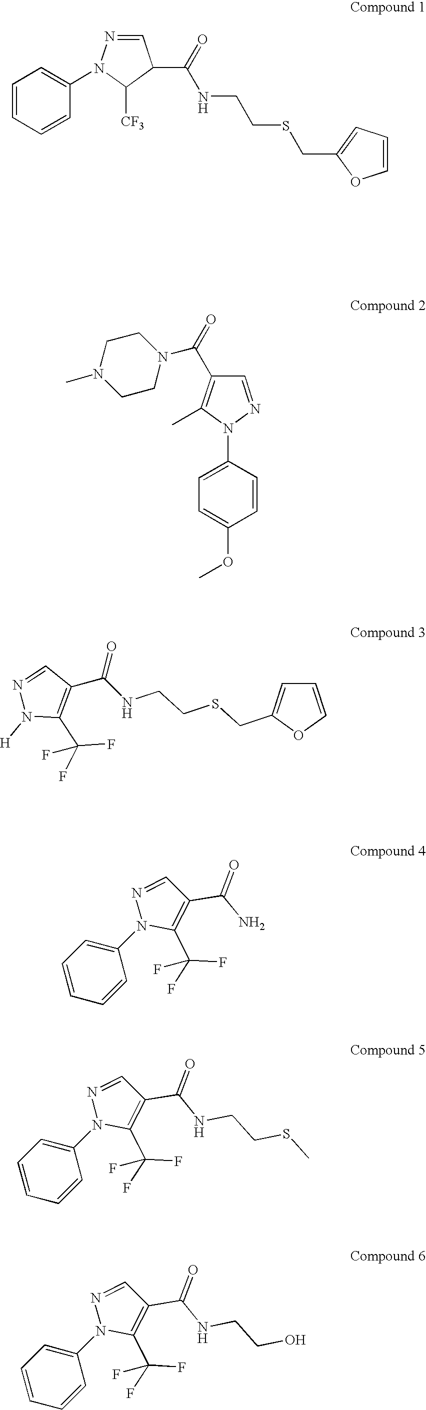

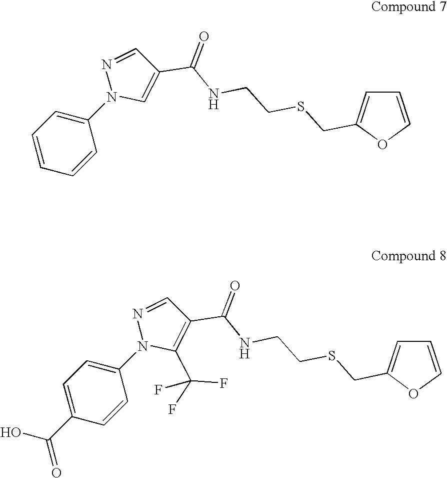

Examples of the synthesis of compounds 3, 5, 6, 7 and 8 are given below.

### Synthesis of Compound 3

### Synthesis of ethyl 5-trifluoromethyl-1H-pyrazole-4-carboxylate

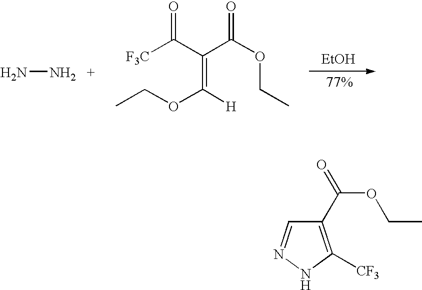

Reagents:

Ethyl 2-(ethoxymethylene-4,4,4-trifluoro)-3-oxobutyrate

| | | | |
|---|---|---|---|
| C₉H₁₁F₃O₄ | MW: 240.18 | m = 4.49 g | 18.71 mmol/1 eq. |

Hydrazine (1M tetrahydrofuran)

| | | |
|---|---|---|
| H₄N₂ | V = 18.71 ml | 18.71 mmol/1 eq. |
| Ethanol | V = 25 ml | |

Procedure:

A 1 M solution (THF) of hydrazine is added to 25 ml of ethanol in a 100 ml three-necked reactor, under argon and with magnetic stirring. The suspension is cooled to −15° C. (CCl₄/N₂ bath) and the oxobutyrate is added dropwise to the hydrazine over 30 minutes.

After 2 hours 30 minutes at room temperature, since no change is visible by thin layer chromatography, the reaction medium is heated at the reflux point of the ethanol (EtOH) for 16 hours. Once cooled to room temperature, the solvent is then evaporated off and the solid obtained is washed twice with 10 ml of pentane and filtered through a sinter funnel.

3 g of a crystalline white solid are thus recovered. (Yield: 77%).

Analyses:

Beige-coloured solid, of which the structure obtained is in accordance (¹H NMR) (¹³C NMR).

### Synthesis of 5-trifluoromethyl-1H-pyrazole-4-carboxylic acid

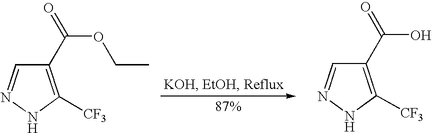

Reagents:

| Ethyl 5-trifluoromethyl-1H-pyrazole-4-carboxylate | | | |
|---|---|---|---|
| C₇H₇N₂O₂F₃ | MW: 208.14 | m = 2.97 g | 14.3 mmol/1 eq. |
| Sodium hydroxide | | | |
| NaOH | MW: 39.99 | m = 5.71 g | 142 mmol/10 eq. |
| Ethanol | | V = 30 ml | |

Procedure:

The pyrazole is dissolved in ethanol in a 250 ml reactor with magnetic stirring. After 15 minutes at room temperature, 1.2 N sodium hydroxide solution (120 ml of water) is added. The reaction medium is then refluxed for 18 hours.

The solution is then cooled to 10° C. and then acidified with 1 N HCl. After evaporating off all of the ethanol and two thirds of the water under reduced pressure, the white precipitate formed is recovered by filtration on a sinter funnel, washed with water and then dried under high vacuum.

The fine white powder (2.30 g) obtained is characterized and corresponds to the expected product (yield: 87%).

Analyses:

White solid, of which the structure obtained is in accordance (¹H NMR) (¹³C NMR).

### Synthesis of N-{2-[(2-furylmethyl)thio]ethyl}-5-trifluoromethyl-1H-pyrazole-4-carboxylic acid

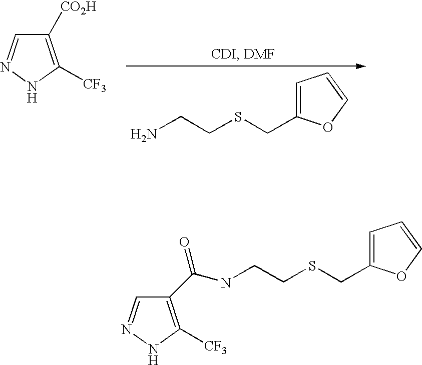

Reagents:

5-Trifluoromethyl-1H-pyrazole-4-carboxylic acid

| | | | |
|---|---|---|---|
| C₅H₃N₂O₂F₃ | MW: 180.08 | m = 0.80 g | 4.44 mmol/1 eq. |

Carbonyldiimidazole (CDI)

| | | | |
|---|---|---|---|
| C₇H₆N₄O | MW: 162.15 | m = 0.815 g | 5.39 mmol/1.4 eq. |

2-(Furfuryithio) ethylamine

| | | | |
|---|---|---|---|
| C₇H₁₁NOS | MW: 157.234 | m = 3.22 g | 21.9 mmol/4.6 eq. |
| Dimethylformamide | | V = 10 ml | |

Procedure:

The pyrazole is dissolved in DMF in a 100 ml reactor under nitrogen and with magnetic stirring. CDI is then added quickly in a single portion and the mixture is kept stirring for about 45 minutes. A persistent insoluble product is observed. The amine is then added rapidly dropwise by syringe. After stirring overnight, the reaction medium is quenched in 100 ml of an ice/water mixture. The white precipitate formed is recovered by filtration and the filtrate is extracted with ethyl acetate (2×25 ml). The organic phase is combined with the precipitate and concentrated to dryness.

This crude mixture is then chromatographed on silica gel (flash chromatography, elution with 3/1 hexane/ethyl acetate, 1% aqueous ammonia). The fraction corresponding to the expected product (Rf: 0.45 in CH₂Cl₂/1% NH₃) is then isolated and concentrated to dryness. The oil obtained is taken up in 2 ml of ethanol and quenched in 100 ml of an ice/water mixture. The precipitate obtained is recovered by filtration, filtered through a sinter funnel and concentrated to dryness.

402 mg of a white solid are thus obtained (yield: 30%).

Analyses:

White solid.

¹H NMR: (DMSO); 7.93 (s, 1H, CH pyrazole), 7.26 (s, 1H, CH furyl), 7.19 (m, 2H, NH+CONH), 6.10 (d, 2H, H furyl), 3.61 (s, 2H, CH₂), 3.37 (q, 2H, CH₂), 2.43 (t, 2H, CH₂).

¹³CNMR: (DMSO/CDCl₃); 158.43 (CONH), 143.1 (CH), 111.4 (2CH), 108.7 (CH), 39.3 (CH₂), 32.1 (CH₂), 28.8 (CH₂). Quaternary carbons not visible due to the small amount of compound in the tube.

### Synthesis of Compounds 5 and 6

### Synthesis of ethyl 5-methyl-1-phenyl-1H-pyrazole-4-carboxylate

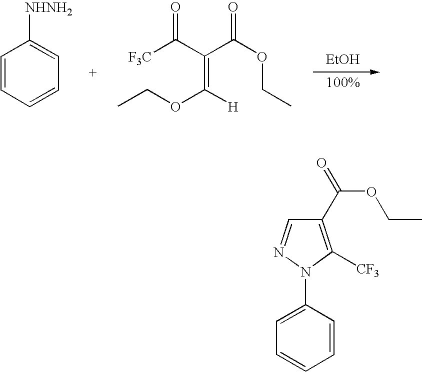

Reagents:

| Ethyl 2-(ethoxymethylene-4,4,4-trifluoro-3-oxobutyrate | | | |
|---|---|---|---|
| C₉H₁₁F₃O₄ | MW: 240.18 | m = 15.00 g | 62 mmol/1 eq. |
| Phenylhydrazine | | | |
| C₇H₈N₂O₂ | MW: 108.14 | m = 6.38 g | 60 mmol/1.1 eq. |
| Ethanol | | V = 500 ml | |

Procedure:

The phenylhydrazine is suspended in 500 ml of absolute ethanol in a 1 L three-necked reactor, under argon and with magnetic stirring. The solution is cooled to −15° C. (CCl₄/N₂ bath) and the oxobutyrate is added dropwise to the hydrazine over 45 minutes. After 4 hours at room temperature, the solution is concentrated to dryness. The yellow powder obtained is washed with pentane and dried under vacuum, and 18.5 g of a white solid are thus isolated (yield: >100%).

Analyses:

White solid, of which the structure obtained is in accordance (¹H NMR) (¹³C NMR).

### Synthesis of ethyl 5-methyl-1-phenyl-1H-pyrazole-4-carboxylate

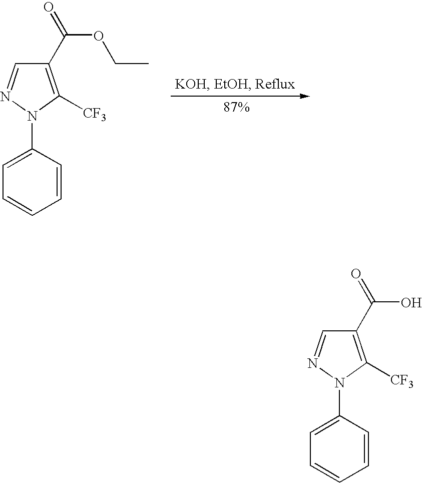

Reagents:

Ethyl 5-trifluoromethyl-1-phenyl-1H-pyrazole-4-carboxylate

| | | | |
|---|---|---|---|
| C₂₁H₁₇N₂O₄F₃ | MW: 418.37 | m = 18.5 g | 65 mmol/1 eq. |

Potassium hydroxide (85%)

| | | | |
|---|---|---|---|
| KOH | MW: 56.11 | m = 6.43 g | 97.5 mmol/1.5 eq. |
| Ethanol | | V = 150 ml | |

Procedure:

The pyrazole is added to a solution of potassium hydroxide in ethanol in a 250 ml reactor under magnetic stirring. After 15 minutes at room temperature, the reaction medium is then refluxed for 3 hours. Once cooled to room temperature, the solution is added to 600 ml of water. The mixture is washed 3 times with 250 ml of ether. The aqueous phase is acidified with 37% HCl to pH 1. The residual ethanol is evaporated off and a yellow precipitate appears in solution. The precipitate is filtered off on a sinter funnel, washed with water and dried under high vacuum for 72 hours.

14.5 g of a yellow powder are thus obtained (yield: 87%).

Analyses:

Yellow solid, of which the structure obtained is in accordance (¹H NMR) (¹³C NMR).

### Synthesis of N-(2-methylthioethyl)-1-phenyl-5-tri-fluoromethyl-1H-pyrazole-4-carboxamide (Compound 5)

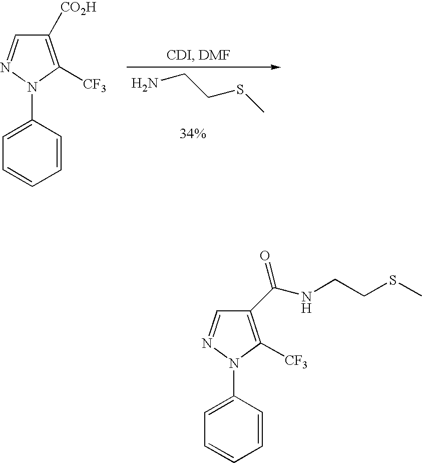

Reagents:

1-Phenyl-5-trifluoromethyl-1H-pyrazolecarboxylic acid

| | | | |
|---|---|---|---|
| C₁₀H₈N₂O₂ | MW: 188.19 | m = 1.20 g | 4.71 mmol/1 eq. |

Carbonyldiimidazole (CDI)

| | | | |
|---|---|---|---|
| C₇H₆N₄O | MW: 162.15 | m = 0.88 g | 5.42 mmol/1.15 eq. |

2-Thiomethylethylamine

| | | | |
|---|---|---|---|
| C₃H₉NS | MW: 91.18 | m = 2.00 g | 21.97 mmol/4.7 eq. |
| DMF | | V = 8.3 ml | |

Procedure:

The pyrazole is dissolved in DMF in a reactor under nitrogen and with magnetic stirring. CDI is then added quickly in a single portion and the mixture is kept stirring for about 20 minutes. The amine is then added quickly dropwise by syringe. After stirring for 3 hours, monitoring of the reaction by tlc indicates the disappearance of all of the starting material.

The reaction medium is then quenched in 80 ml of an ice/water mixture. The white precipitate formed after stirring for 15 minutes is then recovered by filtration on a sinter funnel and dried by suction. The orange-coloured solid obtained is then taken up in 50 ml of ethyl ether, washed once with water and dried over sodium sulphate. After filtration through a sinter funnel and evaporation under partial vacuum, 0.8 g of a yellow solid is thus obtained. After taking up again in 50 ml of dichloromethane and then drying over sodium sulphate, the product is chromatographed on silica (elution: 7/3 hexane/ethyl acetate) and then recrystallized from toluene to give 530 mg of a white solid. (Yield: 34%) characterized by NMR in monohydrate form (peak at 1.6 ppm).

Analyses:

White solid.

NMR ¹H: (CDCl₃);_{—}7.93 (s, 1H, H pyrazole), 7.52-7.32 (m, 5H, H arom.), 6.35 (m, 1H, NH), 3.68 (q, 2H, CH₂), 2.72 (t, 2H, CH₂), 2.15 (s, 3H, CH₃).

NMR ¹³C: (CDCl₃); 161.7 (CO—NH), 140.0 (CH arom.), 139.6 (C arom.), 130.3 (CH arom.), 129.8 (C), 129.6 (2 CH arom.), 126.2 (2 CH arom.), 121.2 (C), 119 (not visible, CF₃), 38.3 (CH₂), 34.0 (CH₂), 15.2 (CH₃).

### Synthesis of N-(2-hydroxyethyl)-1-phenyl-5-trifluoromethyl-1H-pyrazole-4-carboxamide (Compound 6)

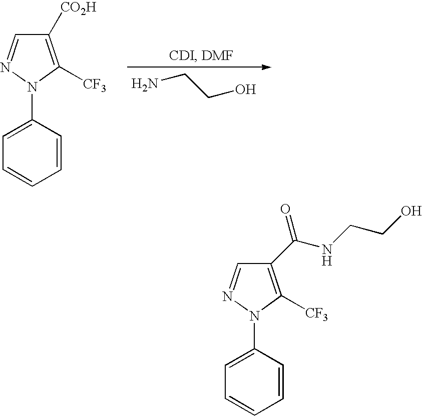

Reagents:

1-Phenyl-5-trifluoromethyl-1H-pyrazolecarboxylic acid

| | | | |
|---|---|---|---|
| C₁₀H₈N₂O₂ | MW: 188.19 | m = 0.83 g | 4.41 mmol/1 eq. |

Carbonyldiimidazole (CDI)

| | | | |
|---|---|---|---|
| C₇H₆N₄O | MW: 162.15 | m = 0.82 g | 5.05 mmol/1.15 eq. |

2-Ethanolamine

| | | | |
|---|---|---|---|
| C₂H₇NO | MW: 61.08 | m = 1.34 g | 20.5 mmol/4.7 eq. |
| DMF | | V = 10 ml | |

Procedure:

The pyrazole is dissolved in DMF in a reactor under nitrogen and with magnetic stirring. CDI is then added quickly in a single portion and the mixture is kept stirring for about 20 minutes. The amine is then added rapidly dropwise by syringe. After stirring for 3 hours, monitoring of the reaction by tlc indicates the disappearance of all of the starting material.

The reaction medium is then quenched in 75 ml of an ice/water mixture. The white precipitate formed after stirring for 15 minutes is then recovered by filtration on a sinter funnel and dried by suction. The orange-coloured solid obtained is then taken up in 50 ml of dichloromethane, washed once with water and dried over sodium sulphate. After filtration through a sinter funnel and evaporation under partial vacuum, 1.1 g of a yellow solid are thus obtained.

This solid is taken up in a minimum amount of toluene, but the product is found to be partially soluble in the solvent. The product is thus recrystallized under cold conditions to result in the formation of 470 mg of a white solid (yield: 33%) characterized in the form of a derivative combined with 0.5 molecule of water.

Analyses:

White solid.

¹H NMR: (CDCl₃);_{—}7.93 (s, 1H, H pyrazole), 7.60-7.20 (m, 5H, H arom.), 6.48 (m, 1H, NH), 3.85 (t, 2H, CH₂), 3.63 (t, 2H, CH₂), 2.00 (broad s, 2H, OH+H₂O).

¹³C NMR: (CDCl₃); 162.4 (CO—NH), 140.0 (CH arom.), 139.4 (C arom.), 130.4 (CH arom.), 130.3 (C), 129.6 (2 CH arom.), 126.2 (2 CH arom.), 121.1 (C), 119.8 (q, CF₃).

### Synthesis of Compound 7

### Synthesis of 1-phenyl-1H-pyrazolecarbaldehyde

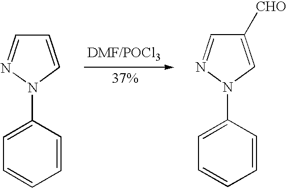

Reagents:

1-Phenyl-1H-pyrazole

| | | | |
|---|---|---|---|
| C₂H₃N [75-05-8] | MW: 41.05 | V = 39.9 ml | 77 mmol/1 eq. |

DMF

| | | |
|---|---|---|
| C₂H₆O [64-17-5] | MW: 46.06 | V = 1.1 L |

Phosphorus oxychloride

| | | | |
|---|---|---|---|
| [7803-49-8] | MW: 33.03 | V = 100 ml | 1.63 mmol/2.13 eq. |

Procedure: (Vilsmeier-type Formylation Reaction)

10 ml of DMF are placed in a 100 ml reactor, under nitrogen and with magnetic stirring, and are immediately cooled to 0° C. using an ice/water bath. The phosphorus oxychloride is added dropwise by syringe over 12 minutes. After 1 hour at 0° C., a solution of 1-phenylpyrazole (in 10 ml of DMF) is added over 2 minutes using a syringe, by rapid dropwise addition. After a further 5 minutes at 0° C., the mixture is warmed to room temperature over 15 minutes and then maintained at 100° C. for 2 hours 30 minutes. Total disappearance of the starting material is observed by tlc (9/1 hexane/acetic acid (EtOAc); Rf: 0.35). Once cooled to room temperature, the reaction medium is added cautiously to 20 g of ice-water in a fume cupboard.

After stirring for 18 hours, the mixture is extracted twice with 250 ml of ethyl acetate. The combined organic phases are dried over sodium sulphate, filtered on a sinter funnel and evaporated to dryness, and the residue is then filtered through a pad of silica on a sinter funnel (elution: pure hexane, 8/2 hexane/CH₂Cl₂, 1/1 hexane/CH₂Cl₂, 100% CH₂Cl₂). The purely chlorinated fractions allow the isolation of 1.17 g of a yellow oil, which crystallizes spontaneously once taken up in hexane. A second fraction (eluted with 1/1 hexane/CH₂Cl₂), taken up in hexane, also allows the isolation of a solid. The solids are combined and washed three times with 10 ml of hexane to give 2.21 g of a white solid (yield: 37%).

Analyses:

White solid.

tlc: (pure dichloromethane): Rf: 0.05 (UV), ¹H NMR, ¹³C NMR.

### Synthesis of 1-phenyl-1H-pyrazolecarboxylic acid

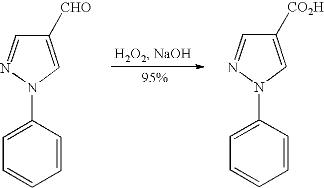

Reagents:

| 1-Phenyl-1H-pyrazolecarbaldehyde | | | |
|---|---|---|---|
| C₁₀H₈N₂O | MW: 172.18 | m = 2.12 g | 12.31 mmol/1 eq. |
| H₂O₂ (30% aq.) | | | |
| H₂O₂ | MW: 34.01 | m = 8.02 g | 70.6 mmol/6.1 eq. |
| Sodium hydroxide | | | |
| NaOH | MW: 40.0 | m = 1.04 g | 26 mmol/2.1 eq. |

Procedure:

The sodium hydroxide is dissolved in 20 ml of water in a 100 ml three-necked reactor, under nitrogen and with magnetic stirring. The pyrazole is then added in a single portion. A persistent insoluble product is observed, even at a temperature of 45-50° C. The aqueous hydrogen peroxide solution is added to the suspension in 6 portions over 50 minutes. After 5 hours at 50° C., monitoring by tlc reveals the persistence of a large portion of substrate. 10 ml of 1 N NaOH (0.4 g of NaOH) and 5 g of aqueous hydrogen peroxide solution are added. After stirring for a further 1 hour at 50° C., the insoluble product has totally disappeared and monitoring by tlc reveals the consumption of all of the starting material (revelation: dinitrophenylhydrazine).

The reaction medium, cooled to room temperature, is then added to 150 ml of an ice/2N HCl mixture (2/1). The white precipitate formed is filtered off on a Büchner funnel, after stirring for 30 minutes, and is washed three times with water. After redissolving in 250 ml of ethyl acetate, drying over MgSO₄ and then filtering and evaporating to dryness, 2.25 g of a white solid are thus isolated (yield: 96%).

Analyses:

White solid, of which the structure obtained is in accordance (¹H NMR) (NMR).

### Synthesis of N-{2-[(2-furylmethyl)thio]ethyl}-1-phenyl-1H-pyrazole-4-carboxamide

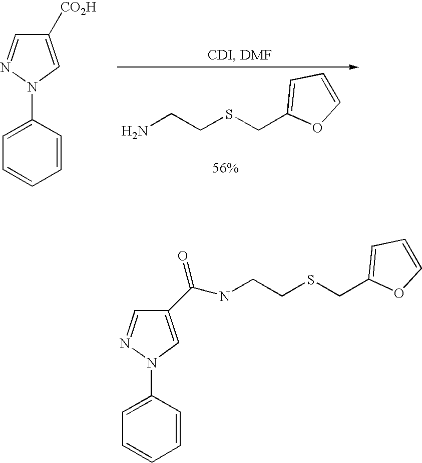

Reagents:

1-Phenyl-1H-pyrazolecarboxylic acid

| | | | |
|---|---|---|---|
| C₁₀H₈N₂O₂ | MW: 188.19 | m = 0.83 g | 4.41 mmol/1 eq. |

Carbonyldiimidazole (CDI)

| | | | |
|---|---|---|---|
| C₇H₆N₄O | MW: 162.15 | m = 0.82 g | 5.05 mmol/1.15 eq. |

2-(Furfurylthio)ethylamine

| | | | |
|---|---|---|---|
| C₇H₁₁NOS | MW: 157.234 | m = 3.23 g | 20.5 mmol/4.7 eq. |
| DMF | | V = 10 ml | |

Procedure:

The pyrazole is dissolved in DMF in a reactor under nitrogen and with magnetic stirring. CDI is then added quickly in a single portion and the mixture is kept stirring for about 20 minutes. The amine is then added by syringe, via rapid dropwise addition. After stirring for 3 hours 30 minutes, monitoring of the reaction by tlc indicates the disappearance of all of the starting material.

The reaction medium is then quenched in 80 ml of an ice/water mixture. The white precipitate formed after stirring for 15 minutes is then recovered by filtration on a sinter funnel and dried by suction. The orange-coloured solid obtained is then taken up in 50 ml of dichloromethane, washed once with water and dried over sodium sulphate. After filtration through a sinter funnel and evaporation under partial vacuum, 0.8 g of a yellow solid is thus obtained.

This solid is chromatographed on silica (elution: 7/3 hexane/ethyl acetate) and then recrystallized from toluene.

0.80 g of a beige-coloured solid is thus recovered. (Yield: 56%). It is characterized in the form of a compound combined with half a molecule of water.

Analyses:

Beige-coloured solid.

tlc (3/7 hexane/EtOAc): Rf=0.70.

¹H NMR: (CDCl₃); 8.38 (s, 1H, CH), 7.95 (s, 1H, CH), 7.72 (m, 2H, H arom.), 7.45 (m, 2H, H arom.), 7.36 (m, 2H), 6.31 (m, 3H, 2CH+NH), 3.78 (s, 2H, S—CH₂), 3.60 (q, 2H, N—CH₂), 2.78 (t, 2H, CH₂—S).

¹³C NMR: (CDCl₃); 162.5 (CO), 151.6 (C), 14.5 (CH), 139.7 (2 CH), 139.4 (C), 129.8 (CH), 128.6 (CH), 127.6 (CH), 120.3 (C), 119.7 (CH), 110.8 (CH), 108.1 (CH), 38.2 (CH₂), 31.8 (CH₂), 28.2 (CH₂).

### Synthesis of Compound 8

### Synthesis of 4-[4-(ethoxycarbonyl)-5-methyl-1H-pyrazol-1-yl]benzoic acid

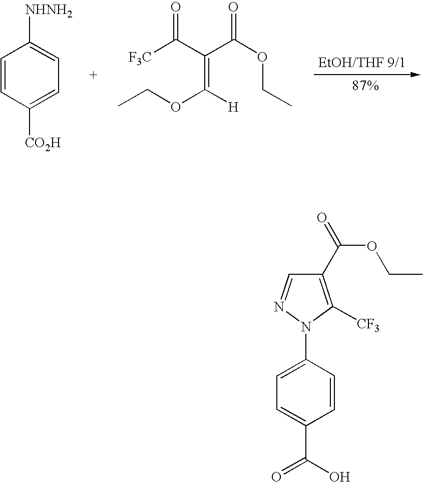

Reagents:

Ethyl 2-(ethoxymethylene-4,4,4-trifluoro-3-oxobutyrate

| | | | |
|---|---|---|---|
| C₉H₁₁F₃O₄ | MW: 240.18 | m = 6.50 g | 27.07 mmol/1 eq. |

4-Hydrazinobenzoic acid

| | | | |
|---|---|---|---|
| C₇H₈N₂O₂ | MW: 152.17 | m = 4.12 g | 27.07 mmol/1 eq. |
| Ethanol | | V = 90 ml | |
| THF | | V = 10 ml | |

Procedure:

The hydrazinobenzoic acid is suspended in 90 ml of absolute ethanol in a 250 ml three-necked reactor, under argon and with magnetic stirring. 10 ml of THF are added to promote the dissolution of the reagent (without success). The suspension is cooled to −15° C. (CCl₄/N₂ bath) and the oxobutyrate is added to the hydrazine dropwise over 30 minutes. After 2 hours 30 minutes at room temperature, the solution has become totally clear (a single spot visible on tlc with characteristic revelation of pyrazoles by UV at 254 nm). The solvent is then evaporated off and the yellow solid obtained is washed twice with 20 ml of pentane and filtered through a sinter funnel. The yellow powder obtained is dried under vacuum and 7.70 g of a beige-coloured solid are thus isolated (yield: 87%).

Analyses:

Beige-coloured solid, of which the structure obtained is in accordance (tlc, ¹H NMR, ¹³C NMR).

### Synthesis of 1-(4-carboxyphenyl)-5-trifluoromethyl-1H-pyrazole-4-carboxylic acid

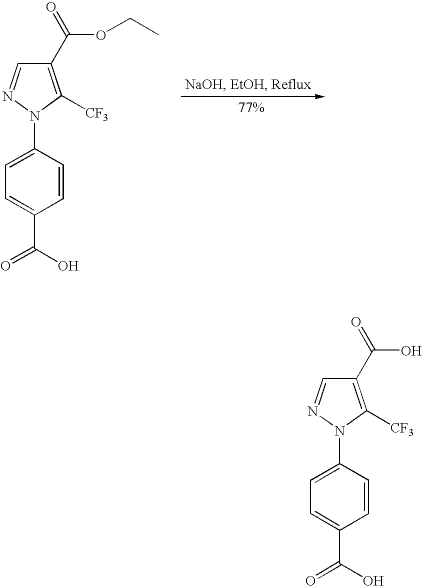

Reagents:

4-[4-(ethoxycarbonyl)-5-methyl-1H-pyrazol-1-yl]benzoic
acid

| | | | |
|---|---|---|---|
| C₁₄H₁₁N₂O₄F₃ | MW: 328.24 | m = 3.25 g | 9.9 mmol/1 eq. |

Sodium hydroxide

| | | | |
|---|---|---|---|
| NaOH | MW: 39.99 | m = 1.38 g | 150 mmol/15 eq. |
| Ethanol | | V = 30 ml | |

Procedure:

The pyrazole is dissolved in ethanol in a 100 ml reactor (equipped with a condenser) under magnetic stirring. After 15 minutes at room temperature, a solution of sodium hydroxide (1.38 g in 50 ml of water) is added. The reaction medium is stirred at room temperature for 5 minutes and then refluxed for 13 hours.

The mixture is then cooled to room temperature and acidified with 3 N HCl solution. The white precipitate obtained is then filtered off on a sinter funnel, rinsed with water and then dried on a rotary evaporator and then with a drying pump.

2.03 g of solid are thus obtained (yield: 77%).

Analyses:

White solid, of which the structure is in accordance (¹H NMR, ¹³C NMR).

### Synthesis of 4-{4-[({2-[(2-furylmethyl)thio]ethyl}-amino)carbonyl]-5-trifluoromethyl-1H-pyrazol-1-yl}-benzoic acid

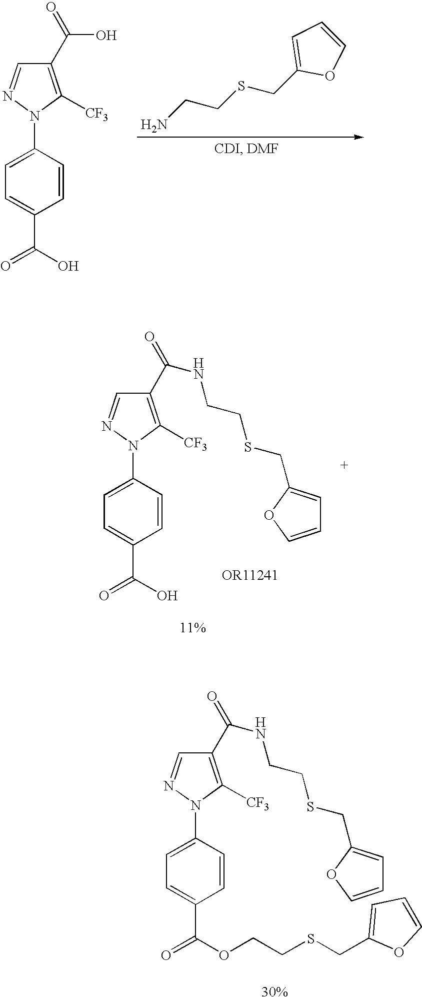

Reagents:

1-(4-carboxyphenyl)-5-trifluoromethyl-1H-pyrazole-4-
carboxylic acid

| | | | |
|---|---|---|---|
| C₁₂H₇N₂O₂F₃ | MW: 268.19 | m = 1.03 g | 3.04 mmol/1 eq. |

Carbonyldiimidazole (CDI)

| | | | |
|---|---|---|---|
| C₇H₆N₄O | MW: 162.15 | m = 0.81 g | 5.0 mmol/1.3 eq. |

2-(furfurylthio) ethylamine

| | | | |
|---|---|---|---|
| C₇H₁₁NOS | MW: 157.23 | m = 2.25 g | 14.3 mmol/4.7 eq. |
| DMF | | V = 12 ml | |

Procedure:

The pyrazole is dissolved in DMF in a reactor under nitrogen and with magnetic stirring. CDI is then added rapidly in a single portion and the mixture is kept stirring for about 30 minutes. The amine is then added by syringe, via rapid dropwise addition. After stirring overnight, monitoring of the reaction by tlc indicates the disappearance of all of the starting material.

The reaction medium is then quenched in 100 ml of an ice/water mixture. The white precipitate formed after stirring for 15 minutes is then recovered by filtration on a sinter funnel and dried by suction. The white solid obtained is then analysed (tic, NMR) and two products are thus identified. This crude mixture is then chromatographed on silica gel (flash chromatography, elution with 2/1 and then 1/1 hexane/ethyl acetate with 1% formic acid).

The first fraction (Rf: 0.65 in pure CH₂Cl₂, 1% HCO₂H) contains 140 mg of the expected product (11% yield). The identification was performed by NMR by analogy with the other compounds synthesized.

The second fraction (Rf: 0.48) contains 500 mg of the product containing 2 amide functions (30% yield).

Analyses:

White solid, of which the structure obtained is in accordance (¹H NMR, ¹³C NMR).

### Example 9

### Demonstration of the 15-PGDH-specific Inhibitory Properties of the Compounds of Formula (I)

1) Test on 15-PGDH

The enzyme 15-PGDH is obtained as described in patent application FR 02/05067 filed in the name of L'Oréal, as a suspension in a medium adjusted to a concentration of 0.3 mg/ml and then blocked at −80° C. For the purposes of the test, this suspension is thawed and stored in ice.

In parallel, a 100 mM, pH 7.4 Tris buffer containing 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier), and 50 μM of prostaglandin E₂ (P4172, Sigma-Aldrich, L'isle D'Abeau Chesne, BP 701, 38297, Saint Quentin Fallavier) is prepared.

0.965 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.035 ml of enzymatic suspension at 37° C. is introduced into the cuvette concomitantly with the recording (corresponding to an increase in the optical density at 340 nm). The maximum reaction rate is recorded.

The test values (containing the compounds (I)) are compared with the control value (without compound (I)); the results indicated represent the percentage of inhibition of 15-PGDH at a concentration of 50 μM.

2) Test on PGF Synthase

The enzyme PGFS is obtained as described in document FR-A-02/05067, at a concentration of 0.5 mg/ml, as a suspension in a suitable medium, and blocked at −80° C. For the purposes of the test, this suspension is thawed and stored in ice.

In parallel, a 100 mM, pH 6.5 Tris buffer containing 20 μM of 9,10-phenanthrenequinone* (P2896, Sigma-Aldrich, L'isle D'Abeau Chesne, BP 701, 38297, Saint Quentin Fallavier) and 100 μM of β-NADPH (N1630, Sigma-Aldrich, L'isle D'Abeau Chesne, BP 701, 38297, Saint Quentin Fallavier) is prepared in a brown flask (protected from light).

*A stock solution with a titre of 1 mM is prepared in absolute ethanol and brought to 40° C.; the flask is placed in an ultrasound cuvette to facilitate the dissolution of the product.

0.950 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.05 ml of enzymatic suspension at 37° C. is introduced into the cuvette concomitantly with the recording (corresponding to a reduction in the optical density at 340 nm). The maximum reaction rate is recorded.

The test values (containing compound (I)) are compared with the control value (without compound (I)); the results indicated represent the percentage of inhibition of PGFS at a concentration of 50 μM.

| | Inhibition | | | | |
|---|---|---|---|---|---|
| | Percentage of | Percentage of | | | |
| | inhibition of | inhibition of | | | |
| | 15-PGDH at a | PGFS at a | | | |
| Com- | concentration | concentration | | | |
| pound | of 50 μM | of 50 μM | IC_{50dh} | IC_{50fs} | Selectivity |
| 1 | 54% | 15% | — | — | — |
| 2 | — | — | 10 μm | >75 μm | >7.5 |

From this table, it is seen that compounds 1 and 2 are indeed 15-PGDH inhibitors. Furthermore, they inhibit 15-PGDH more selectively than PGFS.

The compositions below are obtained via the usual techniques commonly used in cosmetics or pharmaceutics.

### Example 10

### Hair Lotion

| | |
|---|---|
| Compound 1 | 0.80 g |
| Propylene glycol | 10.00 g |
| Isopropyl alcohol qs | 100.00 g |

This lotion is applied to the scalp, once or twice a day, at a rate of 1 ml per application, massaging the scalp gently to help the active agent to penetrate. The head of hair is then dried in the open air. This lotion makes it possible to reduce hair loss and to promote regrowth of the hair.

### Example 11

### Hair Lotion

| | |
|---|---|
| Compound 1 | 1.00 g |
| Propylene glycol | 30.00 g |
| Ethyl alcohol | 40.00 g |
| Water qs | 100.00 g |

This lotion is applied to the scalp, once or twice a day, at a rate of 1 ml per application, massaging the scalp gently to help the active agent to penetrate. The head of hair is then dried in the open air.

This lotion is applied to the scalp, once or twice a day, at a rate of 1 ml per application, massaging the scalp gently to help the active agent to penetrate.

### Example 12

### Hair Lotion

| | |
|---|---|
| Compound 1 | 0.10 g |
| Latanoprost | 0.10 g |
| Propylene glycol | 30.00 g |
| Ethyl alcohol | 40.00 g |
| Water qs | 100.00 g |

### Example 13

### Wax/water Mascara

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound 1 | 1.00% |
| Black pigment | 5.00% |
| Preserving agent qs | |
| Water qs | 100.00% |

This mascara is applied to the eyelashes like a standard mascara with a mascara brush.

1. Use of an effective amount of at least one pyrazolecarboxamide compound of formula (I), or a salt thereof: 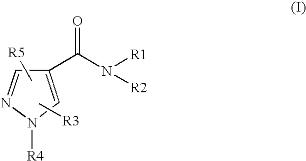
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
as an agent for inducing and/or stimulating the growth of keratin fibres, especially human keratin fibres, and/or for reducing their loss and/or increasing their density.

2. Cosmetic use of at least one pyrazolecarboxamide compound of formula (I), or a salt thereof: 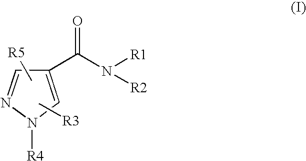
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R′₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
in a cosmetic composition for caring for and/or making up human keratin fibres, to induce and/or stimulate their growth, to reduce their loss and/or to increase their density.

3. Use of at least one pyrazolecarboxamide compound of formula (I), or a salt thereof: 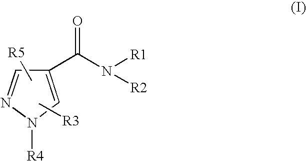
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆) NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
for the preparation of a composition for caring for or treating human keratin fibres, which is intended to induce and/or stimulate the growth of the said fibres and/or to reduce their loss and/or to increase their density.

4. Use of at least one pyrazolecarboxamide compound of formula (I), or a salt thereof: 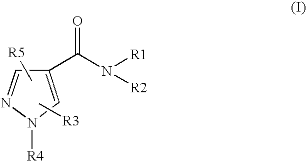
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆) NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
as an inhibitor of 15-hydroxyprostaglandin dehydrogenase, especially of human origin.

5. Use of at least one pyrazolecarboxamide compound of formula (I), or a salt thereof: 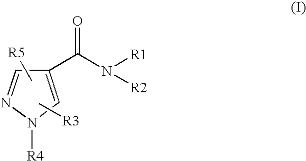
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR′₆R′″₆, SiR₆R′₆R″₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
for the manufacture of a composition for caring for or treating human keratin fibres, which is intended to treat disorders associated with 15-hydroxyprostaglandin dehydrogenase in man.

6. Use according to one of the preceding claims, characterized in that the keratin fibres are head hair, the eyebrows, the eyelashes, beard hair, moustache hair and pubic hair.

7. Use of an effective amount of at least one pyrazolecarboxamide compound of formula (I), or a salt thereof: 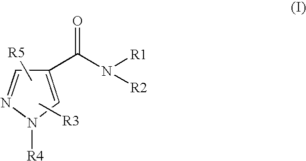
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇So₂R′₇, NR₇C(═NR′₇)NR″₇R′″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆ halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
in a cosmetic composition for caring for human hair, to reduce hair loss and/or to increase hair density and/or to treat alopecia of natural origin.

8. Use of at least one pyrazolecarboxamide compound of formula (I), or a salt thereof: 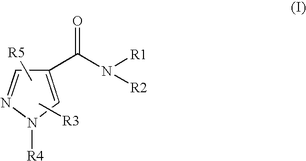
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆
saturated or unsaturated rings, optionally containing at least one hetero atom chosen from O, N and S, these rings containing 4 to 7 atoms and possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
for the preparation of a human hair composition, which is intended to induce and/or stimulate the growth of the hair and/or reduce its loss and/or increase its density and/or treat alopecia of natural origin.

9. Use of at least one pyrazolecarboxamide compound of formula (I), or a salt thereof: 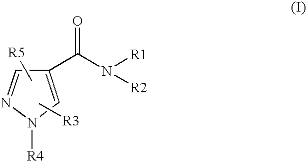
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R′₆,
saturated or unsaturated rings, optionally containing at least one hetero atom chosen from O, N and S, these rings containing 4 to 7 atoms and possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
as a cosmetic composition for caring for and/or making up human eyelashes, to reduce their loss and/or to increase their density.

10. Use of at least one pyrazolecarboxamide compound of formula (I), or a salt thereof: 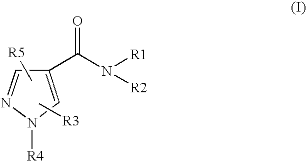
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R₂″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R,
for the preparation of a composition for caring for and/or treating human eyelashes, which is intended to induce and/or stimulate their growth and/or increase their density.

11. Use of at least one pyrazolecarboxamide compound of formula (I), or a salt thereof, for the manufacture of a composition for preserving the amount and/or activity of the prostaglandins in the hair follicles.

12. Cosmetic use of at least one pyrazolecarboxamide compound of formula (I), or a salt thereof, as an agent for preserving the amount and/or activity of prostaglandins in the hair follicles.

13. Use according to one of the preceding claims, characterized in that the pyrazolecarboxamide compound has the formula (II) below, or a salt thereof: 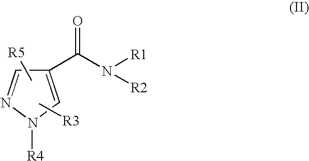
in which:
R₁ and R₂ are chosen independently from:
hydrogen,
saturated or unsaturated, linear or branched C₁-C₂₀ alkyl radicals optionally substituted with at least one substituent T₁, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COOR₆,
saturated or unsaturated rings of 4 to 7 atoms, optionally containing at least one hetero atom chosen from O, N and S, these rings possibly being fused and/or possibly being substituted with at least one substituent T₃ chosen from A and R;
R₄ is chosen from:
hydrogen,
A,
the groups COR₆ and COOR₆,
saturated or unsaturated hydrocarbon-based rings of 4 to 7 atoms, these rings possibly being substituted with at least one substituent T₄ chosen from A and R;
R₆ and R′₆ are chosen from:
hydrogen,
saturated or unsaturated, linear or branched C₁-C₂₀ alkyl radicals optionally substituted with at least one substituent R′,
saturated or unsaturated rings of 4 to 7 atoms, optionally containing at least one hetero atom chosen from O, N and S, these rings possibly being fused and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃ and COOR₇;
R′ is chosen from:
saturated or unsaturated, linear or branched C₁-C₂₀ alkyl radicals,
halogens,
the groups OR₇, SR₇, NR₇R′₇, CN, CF₃ and COOR₇,
saturated or unsaturated rings of 4 to 7 atoms, optionally containing at least one hetero atom chosen from O, N and S, these rings possibly being fused;
R₇ and R′₇ independently represent hydrogen or a saturated or unsaturated, linear or branched C₁-C₂₀ alkyl radical;
A represents a saturated or unsaturated, linear or branched C₁-C₂₀ alkyl radical optionally substituted with at least one substituent T₅ chosen from halogens, the groups OR₇, SR₇, NR₇R′₇, CN, CF₃ and COOR₇ and saturated or unsaturated rings of 4 to 7 atoms optionally containing at least one hetero atom chosen from O, N and S, these rings possibly being fused and/or possibly being substituted with at least one substituent R;
T₁ is chosen from OR₆, SR₆, NR₆R′₆, CN, CF₃ and COOR₆, halogens, saturated or unsaturated rings of 4 to 7 atoms optionally containing at least one hetero atom chosen from O, N and S, these rings possibly being fused and possibly being substituted with at least one substituent R.

14. Use according to one of the preceding claims, characterized in that at least one from among R₁ and R₂ represents a group (CH₂)ₙR₈ with R₈ representing OH or —S—(CH₂)ₘR₉, with R₉ representing H or Hy, in which Hy represents a heterocycle of 4 to 7 atoms.

15. Use according to one of the preceding claims, characterized in that R₁ represents hydrogen and R₂ represents a group (CH₂)ₙR₈ with n being equal to 2 and m being equal to 1.

16. Use according to the preceding claim, characterized in that Hy represents a 5-atom heterocycle.

17. Use according to one of claims 14 to 16, characterized in that Hy comprises oxygen as hetero atom.

18. Use according to one of the preceding claims, characterized in that R₄ represents a hydrocarbon-based ring containing 5 or 6 atoms and especially an optionally substituted phenyl radical.

19. Use according to one of the preceding claims, characterized in that at least one from among R₃ and R₅ represents CF₃.

20. Use according to one of the preceding claims, characterized in that R₃ represents CF₃ and R₅ represents H.

21. Use according to one of the preceding claims, characterized in that the salt of the compound of formula (I) is a salt chosen from the sodium or potassium salts, the zinc (Zn²⁺), calcium (Ca²⁺), copper (Cu²⁺), iron (Fe²⁺), strontium (Sr²⁺), magnesium (Mg²⁺), manganese (Mn²⁺) and ammonium salts, the triethanolamine, monoethanolamine, diethanolamine, hexadecylamine, N,N,N′,N′-tetrakis(2-hydroxypropyl)ethylenediamine and tris(hydroxymethylamino)methane salts, hydroxides, carbonates, halides, sulphates, phosphates and nitrates.

22. Use according to one of the preceding claims, characterized in that the compound satisfies one of the following formulae:
Compound 1

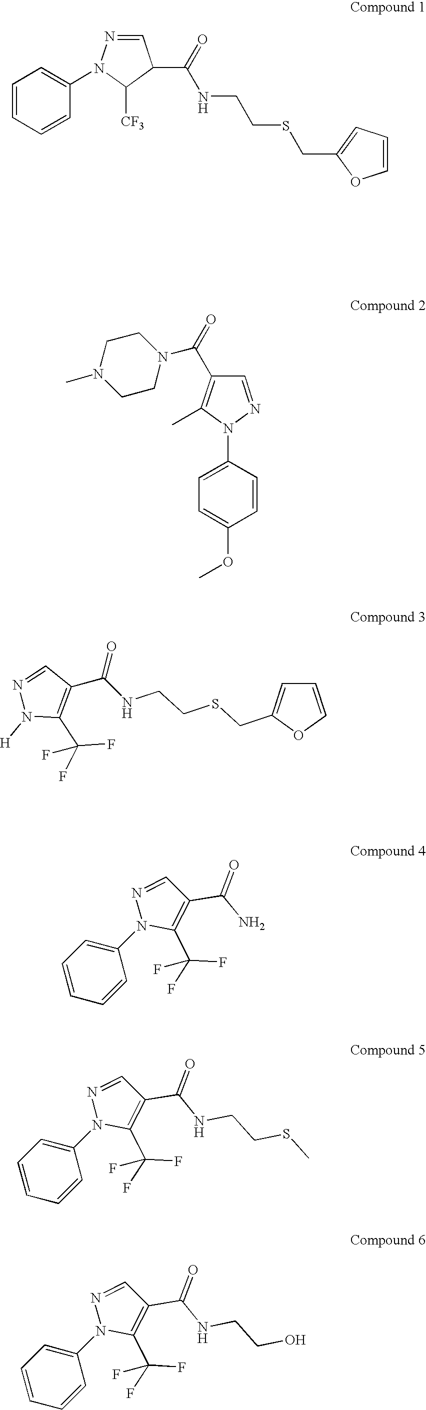

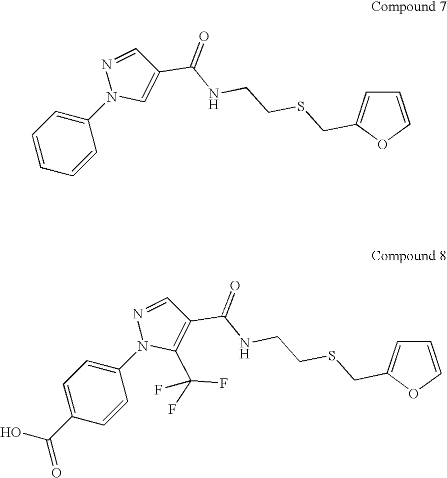

23. Use according to one of the preceding claims, characterized in that the compound of formula (I) or a mixture of compounds of formula (I) is used at a concentration ranging from 10⁻³ to 10%, and preferably from 10⁻² to 2%, relative to the total weight of the composition.

24. Use according to one of the preceding claims, characterized in that the composition is a composition for topical application.

25. Composition for caring for or making up keratin fibres, containing a physiologically acceptable medium and an effective amount of at least one pyrazolecarboxamide compound of formula (I), or a salt thereof: 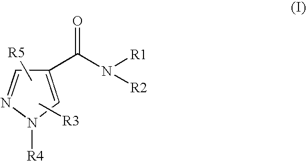
in which:
R₁ and R₂ are chosen independently from:
hydrogen,
saturated or unsaturated, linear or branched C₁-C₂₀ alkyl radicals optionally substituted with at least one substituent T₁,
saturated or unsaturated rings containing at least one hetero atom chosen from O, N and S and saturated hydrocarbon-based rings, these rings containing from 4 to 7 atoms and possibly being fused, comprising a carbonyl or thiocarbonyl function, and/or possibly being substituted with at least one substituent T₂ chosen from A and R, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆)NR″₆R′″₆, SiR₆R′₆R″₆,
saturated or unsaturated rings of 4 to 7 atoms, optionally containing at least one hetero atom chosen from O, N and S, these rings possibly being fused, comprising a carbonyl or thiocarbonyl function, and/or possibly being substituted with at least one substituent T₃ chosen from A and R;
R₄ is chosen from:
hydrogen,
A,
the groups COR₆, CSR₆, COOR₆, CONR₆R′₆, CSNR₆R′₆, SO₂R₆, SO₂NR₆R′₆,
saturated or unsaturated hydrocarbon-based rings, of 4 to 7 atoms, 5-atom heterocycles containing from one to four hetero atoms, 6-atom heterocycles containing from one to three non-adjacent hetero atoms, 4- or 7-atom heterocycles containing from one to three hetero atoms, the hetero atoms being chosen from O, N and S, these heterocycles being saturated or unsaturated, the said rings and the said heterocycles possibly being fused, comprising a carbonyl or thiocarbonyl function, and/or possibly being substituted with at least one substituent T₄ chosen from A and R;
R₆, R′₆, R″₆ and R′″₆ are chosen from:
hydrogen,
saturated or unsaturated, linear or branched C₁-C₂₀ alkyl radicals optionally substituted with at least one substituent R′,
saturated or unsaturated rings, of 4 to 7 atoms, optionally containing at least one hetero atom chosen from O, N and S, these rings possibly being fused, comprising a carbonyl or thiocarbonyl function, and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR′₇, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇;
R′ is chosen from:
saturated or unsaturated, linear or branched C₁-C₂₀ alkyl radicals,
halogens,
the groups OR₇, SR₇, NR₇R′₇, CN, CF₃, COR₇, CSR₇, COOR₇, COSR₇, CSOR₇, CSSR₇, NR₇COR′₇, NR₇CSR7₈, OCOR₇, SCOR₇, CSNR₇R′₇, SO₂R₇, SO₂NR₇R′₇, NR₇SO₂R′₇, NR₇C(═NR′₇)NR″₇R′″₇ and SiR₇R′₇R″₇,
saturated or unsaturated rings, of 4 to 7 atoms, optionally containing at least one hetero atom chosen from O, N and S, these rings possibly being fused and/or comprising a carbonyl or thiocarbonyl function;
R₇, R′₇, R″₇ and R′″₇ independently represent hydrogen or a saturated or unsaturated, linear or branched C₁-C₂₀ alkyl;
A represents a saturated or unsaturated, linear or branched C₁-C₂₀ alkyl radical, optionally substituted with at least one substituent T₅ chosen from: R′ and the saturated or unsaturated rings of 4 to 7 atoms optionally containing at least one hetero atom chosen from O, N and S, these rings possibly being fused, comprising a carbonyl or thiocarbonyl function, and/or possibly being substituted with at least one substituent R;
T₁ is chosen from OR₆, SR₆, NR₆R′₆, CN, CF₃, COR₆, CSR₆, COOR₆, COSR₆, CSOR₆, CSSR₆, NR₆COR′₆, NR₆CSR′₆, OCOR₆, SCOR₆, CSNR₆R′₆, SO₂R₆, SO₂NR₆R′₆, NR₆SO₂R′₆, NR₆C(═NR′₆) NR″₆R′″₆, SiR₆R′₆R″₆, halogens, saturated or unsaturated rings of 4 to 7 atoms optionally containing at least one hetero atom chosen from O, N and S, these rings possibly being fused, comprising a carbonyl or thiocarbonyl function, and possibly being substituted with at least one substituent R.

26. Composition according to claim 25, characterized in that the pyrazolecarboxamide compound has the formula (II) below, or a salt thereof: 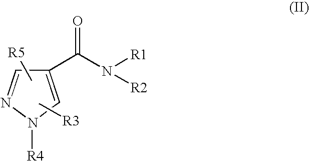
in which:
R₁ and R₂ are chosen independently from:
hydrogen,
saturated or unsaturated, linear or branched C₁-C₂₀ alkyl radicals optionally substituted with at least one substituent T₁, R₁ and R₂ also possibly forming a heterocycle of 4 to 7 atoms with the nitrogen to which they are attached;
R₃ and R₅ are chosen independently from:
hydrogen,
A,
halogens,
the groups OR₆, SR₆, NR₆R′₆, CN, CF₃, COOR₆,
saturated or unsaturated rings of 4 to 7 atoms, optionally containing at least one hetero atom chosen from O, N and S, these rings possibly being fused and/or possibly being substituted with at least one substituent T₃ chosen from A and R;
R₄ is chosen from:
hydrogen,
A,
the groups COR₆ and COOR₆,
saturated or unsaturated hydrocarbon-based rings of 4 to 7 atoms, these rings possibly being substituted with at least one substituent T₄ chosen from A and R;
R₆ and R′₆ are chosen from:
hydrogen,
saturated or unsaturated, linear or branched C₁-C₂₀ alkyl radicals optionally substituted with at least one substituent R′,
saturated or unsaturated rings of 4 to 7 atoms, optionally containing at least one hetero atom chosen from O, N and S, these rings possibly being fused and/or possibly being substituted with at least one substituent R;
R is chosen from:
saturated or unsaturated, linear or branched C₁-C₂₀ alkyl radicals,
halogens,
the groups OR₇, SR₇, NR₇R′₇, CN, CF₃ and COOR₇;
R′ is chosen from:
saturated or unsaturated, linear or branched C₁-C₂₀ alkyl radicals,
halogens,
the groups OR₇, SR₇, NR₇R′₇, CN, CF₃ and COOR₇,
saturated or unsaturated rings of 4 to 7 atoms, optionally containing at least one hetero atom chosen from O, N and S, these rings possibly being fused;
R₇ and R′₇ independently represent hydrogen or a saturated or unsaturated, linear or branched C₁-C₂₀ alkyl radical;
A represents a saturated or unsaturated, linear or branched C₁-C₂₀ alkyl radical optionally substituted with at least one substituent T₅ chosen from halogens, the groups OR₇, SR₇, NR₇R′₇, CN, CF₃ and COOR₇ and saturated or unsaturated rings of 4 to 7 atoms optionally containing at least one hetero atom chosen from O, N and S, these rings possibly being fused and/or possibly being substituted with at least one substituent R;
T₁ is chosen from OR₆, SR₆, NR₆R′₆, CN, CF₃ and COOR₆, halogens, saturated or unsaturated rings of 4 to 7 atoms optionally containing at least one hetero atom chosen from O, N and S, these rings possibly being fused and possibly being substituted with at least one substituent R.

27. Composition according to claim 25 or 26, characterized in that at least one from among R₁ and R₂ represents a group (CH₂)ₙS(CH₂)ₘHy, in which Hy represents a heterocycle.

28. Composition according to one of claims 25 to 27, characterized in that R₁ represents hydrogen and R₂ represents a group (CH₂)ₙS(CH₂)ₘHy in which Hy represents a heterocycle with n being equal to 2 and m being equal to 1.

29. Composition according to one of claims 25 to 28, characterized in that Hy represents a 5-atom heterocycle.

30. Composition according to one of claims 25 to 29, characterized in that Hy comprises oxygen as hetero atom.

31. Composition according to one of claims 25 to 30, characterized in that one from among R₃ and R₅ represents CF₃.

32. Composition according to one of claims 25 to 31, characterized in that R₃ represents CF₃ and R₅ represents H.

33. Composition according to one of claims 25 to 32, characterized in that R₄ represents a hydrocarbon-based ring containing 5 or 6 atoms.

34. Composition according to one of claims 25 to 33, characterized in that the salt of the compound of formula (I) is a salt chosen from the sodium or potassium salts, the zinc (Zn²⁺), calcium (Ca²⁺), copper (Cu²⁺), iron (Fe²⁺), strontium (Sr²⁺), magnesium (Mg²⁺), manganese (Mn²⁺) and ammonium salts, the triethanolamine, monoethanolamine, diethanolamine, hexadecylamine, N,N,N′,N′-tetrakis(2-hydroxypropyl)-ethylenediamine and tris(hydroxymethylamino)methane salts, hydroxides, carbonates, halides, sulphates, phosphates and nitrates.

35. Composition according to one of claims 25 to 36, characterized in that the compound of formula (I) satisfies one of the following formulae:

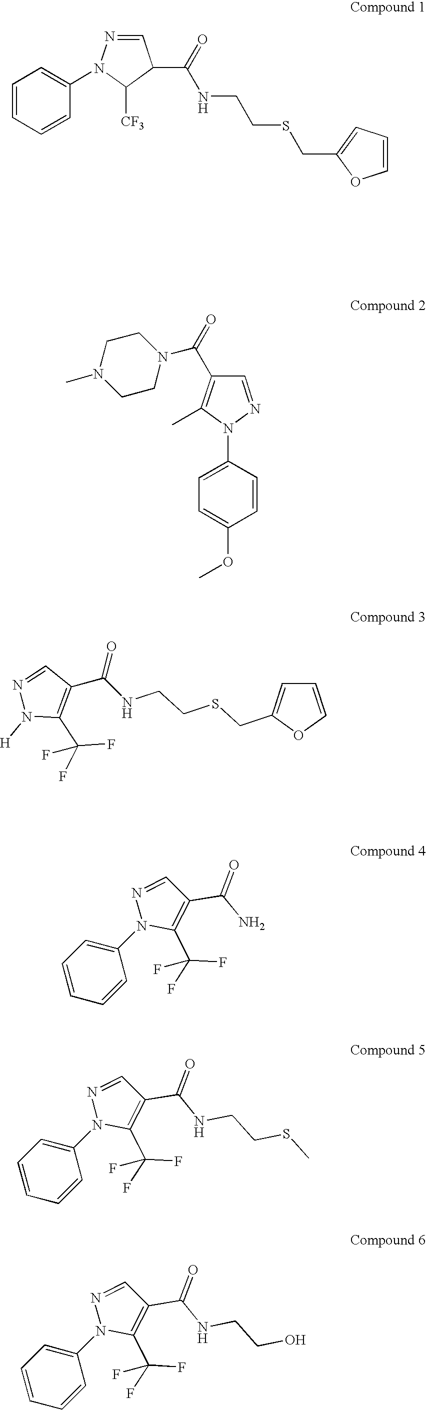

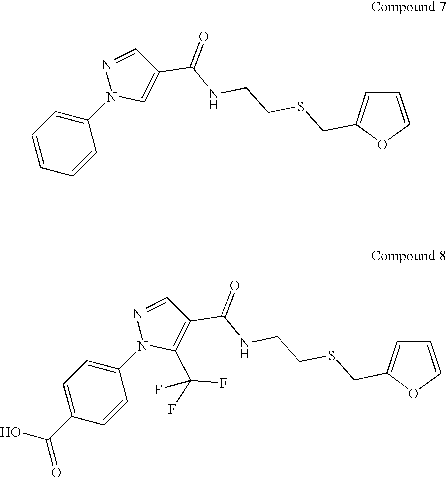

36. Composition according to one of claims 25 to 35, characterized in that the compound of formula (I) is used at a concentration ranging from 10⁻³ to 10%, and preferably from 10⁻² to 2%, relative to the total weight of the composition.

37. Composition according to one of claims 25 to 36, characterized in that the composition is for topical application.

38. Composition according to one of claims 25 to 37, characterized in that it is in the form of a hair cream, a hair lotion, a shampoo, a conditioner or a mascara for the hair or the eyelashes.

39. Composition according to one of claims 25 to 38, characterized in that it is in the form of an aqueous, alcoholic or aqueous-alcoholic solution or suspension.

40. Composition according to one of claims 25 to 39, characterized in that it contains other ingredients chosen from solvents, aqueous-phase or oily-phase thickeners or gelling agents, dyestuffs that are soluble in the medium of the composition, fillers, pigments, antioxidants, preserving agents, fragrances, electrolytes, neutralizers, film-forming polymers, UV-blockers and cosmetic and pharmaceutical active agents other than the compounds of formula (I), and mixtures thereof.

41. Composition according to one of claims 25 to 40, characterized in that it also contains another active agent chosen from proteins, protein hydrolysates, amino acids, polyols, urea, allantoin, sugars and sugar derivatives, plant extracts, hydroxy acids; retinol derivatives, tocopherol derivatives, essential fatty acids, ceramides, essential oils, salicylic acid derivatives, for instance 5-n-octanoyl salicylic acid, hydroxy acid esters, phospholipids and vitamins, and mixtures thereof.

42. Composition according to one of claims 25 to 41, characterized in that it also contains at least one additional active compound that promotes the regrowth and/or limits the loss of keratin fibres.

43. Composition according to one of claims 25 to 42, characterized in that it also contains at least one additional active compound that promotes the regrowth and/or limits the loss of keratin fibres, chosen from aminexil, 6-0-[(9Z,12Z)octadeca-9,12-dienoyl]hexapyranose, lipoxygenase inhibitors, bradykinin inhibitors, prostaglandins and derivatives thereof, prostaglandin receptor agonists or antagonists, non-prostanoic prostaglandin analogues, vasodilators, antiandrogens, cyclosporins and analogues thereof, antimicrobial agents, anti-inflammatory agents, retinoids, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharidic acids, aryl-substituted ethylenes, N-acyl amino acids, flavonoids, ascomycin derivatives and analogues, histamine antagonists, saponins, proteoglycanase inhibitors, oestrogen agonists and antagonists, pseudoterines, cytokines and growth factor promoters, IL-1 or IL-6 inhibitors, IL-10 promoters, TNF inhibitors, benzophenones, hydantoin, octopirox, retinoic acid, antipruriginous agents, antiparasitic agents, antifungal agents, nicotinic acid esters, calcium antagonists, hormones, triterpenes, antiandrogens, steroidal or non-steroidal 5-α-reductase inhibitors, potassium-channel agonists and FP receptor agonists, and mixtures thereof.

44. Composition according to one of claims 41 to 43, characterized in that the additional active compound is chosen from aminexil, FP receptor agonists and vasodilators.

45. Care or makeup composition for keratin fibres, comprising, in a physiologically acceptable medium, in particular a cosmetic medium, at least one compound of formula (I), or a salt thereof, and at least one additional active compound for promoting the regrowth of human keratin fibres and/or for limiting their loss, chosen from aminexil, FP receptor agonists and vasodilators.

46. Composition according to one of claims 42 to 45, characterized in that the additional active compound is chosen from aminexil, minoxidil, latanoprost, butaprost and travoprost.

47. Cosmetic process for treating keratin fibres and/or the skin from which the said fibres emerge, characterized in that it consists in applying to the fibres and/or the skin a cosmetic composition as defined in any of claims 25 to 46, leaving this composition in contact with the fibres and/or the skin, and optionally rinsing it off.

48. Cosmetic care and/or makeup process for human eyelashes, to improve their condition and/or appearance, characterized in that it consists in applying to the eyelashes and/or the eyelids a mascara composition comprising at least one compound of formula (I) or a salt thereof, and leaving this composition in contact with the eyelashes and/or the eyelids.

49. Cosmetic care process for human hair and/or the scalp characterized in that it consists in applying to the hair and/or the scalp a cosmetic composition as defined in any one of claims 25 to 46, leaving the composition in contact with the hair and/or the scalp, and optionally rinsing it off.

50. Pyrazolecarboxamide compound of formula (III), or a salt thereof: 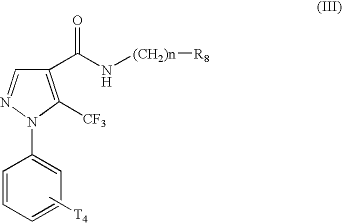
in which R₈ represents OH or —S—(CH₂)ₘ—R₉, with R₉ representing H or Hy; T₄ represents H or 4-COOH; n represents an integer ranging from 1 to 10 and m represents an integer ranging from 1 to 10; Hy represents a heterocycle of 4 to 7 atoms.

51. Compound according to claim 50, characterized in that Hy represents furan.

52. Compound according to claim 50 or 51, characterized in that n=2 and/or m=1.
